SEQUENCE LISTING (12 sequences)

SEQ ID NO: 1 - protein, 14 aa, Artificial Sequence
  Synthetically generated peptide
  DISULFID (1)..(6)
  DISULFID (2)..(10)
  DISULFID (5)..(13)
CCEYCCNPACTGCY

SEQ ID NO: 2 - protein, 19 aa, Artificial Sequence
  Synthetically generated peptide
  DISULFID (6)..(11)
  DISULFID (7)..(15)
  DISULFID (10)..(18)
NSSNYCCEYCCNPACTGCY

SEQ ID NO: 3 - protein, 13 aa, Artificial Sequence
  Synthetically generated peptide
  DISULFID (1)..(6)
  DISULFID (2)..(10)
  DISULFID (5)..(13)
CCEYCCNPACTGC

SEQ ID NO: 4 - protein, 24 aa, Artificial Sequence
  Synthetically generated peptide
  DISULFID (11)..(16)
  DISULFID (12)..(20)
  DISULFID (15)..(23)
AGSAGSAGSGCCEYCCNPACTGCY

SEQ ID NO: 5 - protein, 15 aa, Artificial Sequence
  Synthetically generated peptide
  DISULFID (1)..(6)
  DISULFID (2)..(10)
  DISULFID (5)..(13)
  MISC_FEATURE (15)..(15): Conjugated with Biotin
CCEYCCNPACTGCYC

SEQ ID NO: 6 - protein, 14 aa, Artificial Sequence
  Synthetically generated peptide
  MISC_FEATURE (1)..(1): Conjugated with Biotin
  DISULFID (1)..(6)
  DISULFID (2)..(10)
  DISULFID (5)..(13)
CCEYCCNPACTGCY

SEQ ID NO: 7 - protein, 19 aa, Artificial Sequence
  Synthetically generated peptide
  MISC_FEATURE (1)..(1): Conjugated with Biotin
  DISULFID (6)..(11)
  DISULFID (7)..(15)
  DISULFID (10)..(18)
NSSNYCCEYCCNPACTGCY

SEQ ID NO: 8 - protein, 24 aa, Artificial Sequence
  Synthetically generated peptide
  MISC_FEATURE (1)..(1): Conjugated with Biotin
  DISULFID (11)..(16)
  DISULFID (12)..(20)
  DISULFID (15)..(23)
AGSAGSAGSGCCEYCCNPACTGCY

SEQ ID NO: 9 - protein, 14 aa, Artificial Sequence
  Synthetically generated peptide
  MISC_FEATURE (1)..(1): Conjugated with Fluorescein
  DISULFID (1)..(6)
  DISULFID (2)..(10)
  DISULFID (5)..(13)
CCEYCCNPACTGCY

SEQ ID NO: 10 - protein, 16 aa, Artificial Sequence
  Synthetically generated peptide
  MISC_FEATURE (1)..(1): Conjugated with Fluorescein
  DISULFID (4)..(12)
  DISULFID (7)..(15)
NNDCELCVAVACTGCL

SEQ ID NO: 11 - protein, 24 aa, Artificial Sequence
  Synthetically generated peptide
  MISC_FEATURE (1)..(1): Conjugated with Sulfo-Tag
  DISULFID (11)..(16)
  DISULFID (12)..(20)
  DISULFID (15)..(23)
AGSAGSAGSGCCEYCCNPACTGCY

SEQ ID NO: 12 - protein, 15 aa, Artificial Sequence
  Synthetically generated peptide
  MISC_FEATURE (1)..(1): Conjugated with Fluorescein
  DISULFID (4)..(12)
  DISULFID (7)..(15)
PGTCEICAYAACTGC

(19) United States
(12) Patent Application Publication
(10) Pub. No.: US 2020/0148791 A1
(43) Pub. Date: May 14, 2020

(54) Method of Generating Anti-Linaclotide Antibodies and Uses Thereof

(71) Applicant: Ironwood Pharmaceuticals, Inc., Cambridge, MA (US)

(72) Inventors: Wilmin Bartolini, Amesbury, MA (US); Marco Kessler, Danvers, MA (US)

(21) Appl. No.: 16/407,457

(22) Filed: May 9, 2019

Related U.S. Application Data
(60) Provisional application No. 60/670,252, filed on Apr. 12, 2005.

(51) Int. Cl. C07K 16/44; G01N 33/543; C07K 1/04; C07K 1/22

(52) U.S. Cl. CPC C07K 16/44; G01N 33/54306; C07K 1/042; C07K 1/22; C07K 2317/76; C07K 2317/626; C07K 2317/55; C07K 2317/34; C07K 2317/622

(57) ABSTRACT

The invention provides a method for producing anti-linaclotide antibodies or antigen fragments thereof and uses thereof.

### PRIORITY CLAIM

This application claims priority under 35 U.S.C. § 119(e) to U.S. Provisional Patent Application No. 60/670,252 filed on May 11, 2018, the entire contents of which are hereby incorporated by reference.

### FIELD OF THE INVENTION

This invention relates to the generation and use of anti-linaclotide antibodies.

### SEQUENCE LISTING

This application incorporates by reference in its entirety the Sequence Listing entitled “IW173US1_ST25.txt” (4,514 bytes) which was created on May 8, 2019 and filed electronically herewith.

### BACKGROUND

Linaclotide is a 14-amino acid, orally administered, minimally absorbed peptide that acts on guanylate cyclase C (GC-C) receptor in the gastrointestinal tract. On Aug. 30, 2012, the FDA approved the use of linaclotide for the treatment of irritable bowel syndrome with constipation (IBS-C) and chronic idiopathic constipation (CIC) in adults. It is important to develop and validate assays for the detection of anti-linaclotide antibodies, including IgM, IgG, and IgA, that may be present in the serum at the time of patient sampling.

### SUMMARY

In general, the invention relates to a method of preparing anti-linaclotide antibodies and detecting the presence of linaclotide using anti-linaclotide antibodies.

In one aspect, the invention relates to an antibody or antigen-binding fragment thereof that binds to an epitope of linaclotide.

In another aspect, the invention describes a method for detecting linaclotide in a biological specimen which comprises:

a) contacting the specimen with a first antibody or antigen-binding fragment that binds to an epitope of linaclotide, thereby forming a complex between linaclotide present in the specimen and the first antibody or antigen-binding fragment thereof; and

b) assaying for the presence of the complex.

In another aspect, the invention relates to a method of producing anti-linaclotide antibodies comprising:

a) conjugating linaclotide to a carrier protein;

b) immunizing an animal with the protein conjugated linaclotide to produce an immune response and thereby generate anti-linaclotide antibodies; and

c) harvesting the anti-linaclotide antibodies from the immunized animal.

### BRIEF DESCRIPTION OF THE DRAWINGS

FIG. 1 provides a dot blot analysis (1:5000) of Bleed 3 from immunoreactive rabbits from protocol 1. Lane 1 is reduced linaclotide, lane 2 is native linaclotide, lane 3 is SEQ ID NO: 2, lane 4 is SEQ ID NO: 4. The dots in each lane correspond to varying amounts of peptide per dot (from top to bottom, in μg): 1.0, 0.5, 0.1, 0.05 and 0.01.

FIG. 2 provides a dot blot analysis (1:10,000) of bleeds 6, 9, 12, and 14 from each rabbit from protocol 1. Lane 1 is reduced linaclotide, lane 2 is native linaclotide, lane 3 is SEQ ID NO: 2, lane 4 is SEQ ID NO: 4. The dots in each lane correspond to varying amounts of peptide per dot (from top to bottom, in μg): 1.0, 0.05, 0.1, 0.5 and 0.01.

FIG. 3 provides a dot blot analysis of anti-linaclotide antibodies from protocol 2. Left to right: serum from Protocol 2, (1:4,000; left), 1.0 μg of affinity purified antibody from the same serum pool after caprylic acid IgG enrichment (center), or affinity purified antibodies from protocol 2 without any IgG enrichment (right) were analyzed using the following peptides: lane 1, native linaclotide; lane 2, reduced linaclotide; lane 3, linaclotide that was N-terminally conjugated to BSA; lane 4, AGSA elongated linaclotide peptide; lane 5, NSSN elongated linaclotide peptide. The dots correspond (from top to bottom) to the following amount of peptide (inn): 1.0, 0.5, 0.1, 0.05, and 0.01.

FIG. 4 provides a dot blot analysis of anti-linaclotide antibodies purified from Protocol 1. Serum (1:4,000) or 1.0 μg of affinity purified antibody were analyzed using the following peptides: lane 1, reduced linaclotide; lane 2, native linaclotide; lane 3, AGSA extended linaclotide peptide. The dots correspond (from top to bottom) to the following amount of peptide (in μg): 1.0, 0.5, 0.1, 0.05, and 0.01.

FIG. 5 provides a dot blot analysis of anti-linaclotide antibodies purified from Protocol 2. Serum (1:4,000) or 1.0 μg of affinity purified antibody were analyzed using the following peptides: lane 1, reduced linaclotide; lane 2, native linaclotide; lane 3, AGSA extended linaclotide peptide. The dots correspond (from top to bottom) to the following amount of peptide (in μg): 1.0, 0.5, 0.1, 0.05, and 0.01.

FIG. 6 provides a dot blot analysis of serum (1:4,000) versus affinity purified anti-linaclotide antibodies purified from Protocol 1 from the same rabbits. Two runs were compared to assess the consistency of the antibody after purification. Lane 1, reduced linaclotide; lane 2, native linaclotide; lane 3, SEQ ID NO: 4. The dots correspond (from top to bottom) to the following amount of peptide (in μg): 1.0, 0.5, 0.1, 0.05, and 0.01.

FIG. 7 provides a dot blot analysis of serum (1:4,000) versus affinity purified anti-linaclotide antibodies purified from Protocol 2 from the same rabbits. Two runs were compared to assess the consistency of the antibody after purification. Lane 1, reduced linaclotide; lane 2, native linaclotide; lane 3, SEQ ID NO: 4. The dots correspond (from top to bottom) to the following amount of peptide (in μg): 1.0, 0.5, 0.1, 0.05, and 0.01.

FIG. 8 provides a dot blot analysis of serum (1:1,000) versus affinity purified antibodies (1.0 μg) from rabbits 9515 and 9516 from protocol 3 or rabbit 9520 from protocol 4. Lane 1 is reduced linaclotide, lane 2 is native linaclotide. The dots correspond (from top to bottom) to the following amount of peptide (in μg): 1.0, 0.5, 0.1, 0.05, and 0.01.

FIG. 9 provides dot blots from the N Terminus (A side) and C terminus (B side) of peptides of Table 2.

FIG. 10 provides a comparison of anti-linaclotide antibodies obtained from protocols 1-4 by direct binding ELISA using anti-rabbit IgG-horseradish peroxidase for detection.

FIG. 11 provides a binding plot of anti-linaclotide antibodies obtained by protocols 1 and 2.

FIG. 12 provides an illustration of a bridging assay using fluorescein detection.

FIG. 13 provides an illustration of a Meso-Scale Discovery Assay using a biotinylated linaclotide for antibody capture and SULFO-TAG linaclotide for direct detection of the complex.

FIG. 14 provides results of a checkerboard titration for Meso-Scale Discovery platform optimization for antibody obtained from protocol 1.

FIG. 15 provides results of a checkerboard titration for Meso-Scale Discovery platform optimization for antibody obtained from protocol 2.

FIGS. 16A and 16B provide charts showing the effect of serum dilution on the bridging assay signal for antibodies obtained from protocol 1 in FIG. 16A and antibodies obtained from protocol 2 in FIG. 16B.

FIGS. 17A and 17B provide charts showing the percent variability among individual serum samples in the bridging assay signal for antibodies obtained from protocol 1 in FIG. 16A and antibodies obtained from protocol 2 in FIG. 16B.

FIG. 18 provides a chart showing the displacement of fluorescein labeled linaclotide from antibody obtained from protocol 2 with free linaclotide.

FIG. 19 shows the cross-reactivity by addition of competing unlabeled guanylin, uroguanylin and C-type natriuretic peptide (CNP) against linaclotide using antibodies obtained from protocol 2.

FIG. 20 shows the cross-reactivity by direct binding of linaclotide, uroguanylin, parathyroid hormone (PTH) to antibodies produced using protocol 1 and anti-uroguanylin serum.

FIG. 21 shows an illustration of a direct-binding cross-reactivity assay using a linaclotide capture peptide and a uroguanylin or guanylin detection peptide.

FIG. 22 shows the results of a bridging assay for cross-reactivity using 500 ng/mL of positive control antibody produced by protocol 1, 312 ng/mL of SEQ ID NO: 8, and two concentrations (312 and 625 ng/mL) of fluorescein-modified peptides for each set of reactions.

FIG. 23 shows a decision tree for the detection of cross-reactivity and neutralizing activity in confirmed-positive anti-linaclotide antibodies in patient serum.

FIG. 24 shows a representative concentration-response curve for linaclotide stimulation of GC-C mediated cGMP accumulation in T84 cells.

FIG. 25 shows results of testing polyclonal anti-linaclotide antibodies for neutralization of the pharmacological activity of linaclotide using cGMP accumulation.

FIG. 26 shows the results of testing anti-parathyroid hormone antibodies for neutralization of the pharmacological activity of linaclotide using cGMP accumulation.

### DEFINITIONS

As used herein, “antibody” is used in the broadest sense and encompasses various antibody structures, including but not limited to polyclonal antibodies, monoclonal antibodies, humanized antibodies, chimeric antibodies, human antibodies, multispecific antibodies (e.g., bispecific antibodies), and antibody fragments thereof provided that they exhibit the desired antigen-binding activity. The term “antibody” as referred to herein includes whole antibodies and any antigen binding fragments or single chains thereof. An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region (abbreviated herein as C_{L}). The light chain constant region is comprised of one domain, C_{L}. The V_{H} and V_{L} regions can be further subdivided into regions of hypervariability, termed complementarity determining regions (CDR) which are hypervariable in sequence and/or involved in antigen recognition and/or usually form structurally defined loops, interspersed with regions that are more conserved, termed framework regions (FR or FW). Each V_{H} and V_{L} is composed of three CDRs and four FWs, arranged from amino-terminus to carboxy-terminus in the following order: FW1, CDR1, FW2, CDR2, FW3, CDR3, and FW4. The amino acid sequences of FW1, FW2, FW3, and FW4 all together constitute the “non-CDR region” or “non-extended CDR region” of each of the V_{H} or V_{L}.

The terms “anti-linaclotide antibody” and “an antibody that binds to linaclotide” refer to an antibody that is capable of binding linaclotide with sufficient affinity such that the antibody is useful as a diagnostic agent in targeting linaclotide, and/or in some applications modulates the activity of linaclotide. In one embodiment, the extent of binding of an anti-linaclotide antibody to a GC-C agonist/non-linaclotide protein is less than about 20% of the binding of the antibody to linaclotide as measured, e.g., by a competition assay as described herein.

The terms “antigen-binding portion” of an antibody, or “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-display anti-body libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fd fragments, dAb fragments, Fab′-SH, F(ab′)₂; diabodies; triabodies; linear antibodies; single-chain antibody molecules (e.g., scFv); and multispecific antibodies formed from antibody fragments, minimal recognition units consisting of the amino acid residues that mimic the hypervariable region of an antibody (e.g., an isolated complementarity determining region (CDR) such as a CDR3 peptide), or a constrained FR3 CDR3 FR4 peptide.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

The term “chimeric” antibody refers to an antibody in which a portion of the heavy and/or light chain is derived from a particular source or species, while the remainder of the heavy and/or light chain is derived from a different source or species.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “human” antibody refers to an antibody which possesses an amino acid sequence which corresponds to that of an antibody produced by a human and/or has been made using any of the techniques for making human antibodies as disclosed herein. This definition of a human antibody specifically excludes a “humanized” antibody. “Humanized” antibodies comprise non-human antigen binding residues.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fe region or constant region is according to the EU numbering system, also called the EU index, as described in Kabat et al.

The CDRs contribute to the formation of the antigen-binding, or more specifically, epitope binding site of antibodies. The term “epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 4 or 5-12 amino acids in a unique spatial conformation. Antibodies that recognize the same epitope can be verified in a simple immunoassay showing the ability of one antibody to block the binding of another antibody to a target antigen, for example “binning”, has identified the amino acid residues that bind to the antibodies of the disclosure.

The term “paratope” is derived from the above definition of “epitope” by reversing the perspective. Thus, the term “paratope” refers to the area or region on the antibody which specifically binds an antigen, i.e., the amino acid residues on the antibody which make contact with the antigen (linaclotide).

The term “specifically binds,” or the like, means that an antibody or antigen-binding fragment thereof forms a complex with an antigen that is relatively stable under physiological conditions. Specific binding can be characterized by an equilibrium dissociation constant (K_{D}) of about 3000 nM or less (i.e., a smaller K_{D} denotes a tighter binding), about 2000 nM or less, about 1000 nM or less; about 500 nM or less; about 300 nM or less; about 200 nM or less; about 100 nM or less; about 50 nM or less; about 1 nM or less; or about 0.5 nM.

Specific binding for a particular antigen or an epitope can be exhibited, for example, by an antibody having a K_{D} for an antigen or epitope of at least about 1×10⁻⁴ M, at least about 1×10⁻⁵ M, at least about 1×10⁻⁶ M, at least about 1×10⁻⁷ M, at least about 1×10⁻⁸ M, at least about 1×10⁻⁹ M, alternatively at least about 1×10⁻¹⁰ M, at least about 1×10⁻¹¹ M, at least about 1×10⁻¹² M, or greater, where K_{D} refers to a equilibrium dissociation constant of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the antigen or epitope. Also, specific binding for a particular antigen or an epitope can be exhibited, for example, by an antibody having a Kₐ for an antigen or epitope of at least 20-, 50-, 100-, 500-, 1000-, 5,000-, 10,000- or more times greater for the epitope relative to a control, where Kₐ refers to an association rate of a particular antibody-antigen interaction.

The term “neutralizing antibody” includes an antibody that is capable of inhibiting and/or neutralizing the biological activity of linaclotide, for example by blocking binding or substantially reducing binding of linaclotide to its receptor GC-C or and thus inhibiting or reducing the biological effects triggered by the activation of the GC-C receptor.

The term “detection” includes any means of detecting, including direct and indirect detection.

The articles “a” and “an” are used herein to refer to one or to more than one (i.e., at least one) of the grammatical object of the article. By way of example, “an element” means one element or more than one element. Other than in the operating examples, or where otherwise indicated, all numbers expressing quantities of ingredients or reaction conditions used herein should be understood as modified in all instances by the term “about.”

It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. Other features and advantages of the disclosure will be apparent from the following detailed description, drawings, and claims.

### DETAILED DESCRIPTION

A. Antibodies or Antigen-Binding Fragments Thereof that Bind to Linaclotide

Linaclotide is a peptide GC-C agonist that is orally administered for treatment of irritable bowel syndrome with constipation (IBS-c) and chronic constipation (“CC”). In Phase 2b studies for CC, linaclotide reduced constipation, abdominal discomfort, and bloating throughout the four-week treatment period. Orally administered linaclotide acts locally by activating GC-C at the luminal surface; there are no detectable levels of linaclotide seen systemically after oral administration at therapeutic dose levels.

Linaclotide is a 14 amino acid peptide having the sequence Cys₁ Cys₂ Glu₃ Tyrₐ Cys₅ Cys₆ Asn₇Pro₈ Ala₉ Cys₁₀ Thr₁₁ Gly₁₂ Cys₁₃ Tyr₁₄ (SEQ ID NO: 1) with disulfide bonds between Cys₁ and Cys₆, between Cys₂ and Cys₁₀ and between Cys₅ and Cys₁₃.

In some embodiments, the epitope of linaclotide includes the C-terminal tyrosine (Tyr₁₄) of linaclotide. The C-terminal tyrosine of linaclotide is the tyrosine that includes a carboxylic acid terminus that is not bound to any amino acids.

In some embodiments, the epitope of linaclotide includes the amino acid sequence Cys Thr Gly Cys Tyr, corresponding to the five amino acid residues at the C-terminal end of linaclotide.

In some embodiments, antibodies or antigen-binding fragments that bind to an epitope of linaclotide may be conjugated to a detectable label. “Conjugated to a detectable label” refers to a detectable label that is chemically bound, covalently or non-covalently, to an antibody. A “detectable label” refers to a molecular label that may be detected by, for example, spectroscopic, enzymatic or immunological methods. Detectable labels are well known in the art.

In some embodiments, the detectable label is an enzyme. In some embodiments, the detectable label is a radiolabel. Non-limiting examples of radiolabels include molecules containing ¹³C, ³²P or ¹⁵N. In other embodiments, the detectable label is a fluorescent molecule. Non-limiting examples of fluorescent molecules include fluorescein, ethidium bromide, and green fluorescent protein. Fluorescein is a synthetic organic fluorophore with peak excitation at 494 nm and peak emission at 521 nm. In other embodiments, the detectable label is a chemiluminescent molecule. Non-limiting examples of chemiluminescent molecules include SULFO-TAG and other ruthenium-containing molecules such as [Ru(Bpy)₃]²⁺. SULFO-TAG is a commercially available label sold by Meso Scale Discovery (Rockville, Md.) available as an NHS-ester label (Catalog number R91AO-1 Meso Scale Discovery Rockville, Md.). Meso Scale Discovery provides experimental methods and protocols detailing conjugation of the SULFO-TAG label to peptides, the disclosure of such conjugation methods provided by Meso Scale Discovery are incorporated herein by reference in their entireties. In further embodiments, known peptide-based detectable labels such as Cy3, Cy5, His6, Myc-tag, GST-tag, or maltose binding protein are conjugated to antibodies or antigen binding fragments thereof that bind to an epitope of linaclotide.

B. Methods for Detecting Linaclotide

In one aspect, antibodies or antigen binding fragments thereof that bind an epitope of linaclotide may be used for detecting linaclotide in a biological specimen. “Biological specimen” is herein used to indicate a sample taken from an organism. In some embodiments, the biological specimen is taken from a mammal. In some embodiments the biological specimen is taken from a human. In some embodiments, the biological specimen is human plasma. In other embodiments the biological specimen is human serum, intestinal luminal fluid, fecal matter, urine, saliva, tissue or cells. In some embodiments, the specimen is undiluted before the detection assay or diluted before testing, for example, by 4 fold in assay buffer.

In one embodiment, the method for detecting linaclotide comprises: a) contacting the specimen with a first antibody or antigen-binding fragment thereof, wherein the first antibody or antigen binding fragments thereof binds to an epitope of linaclotide, thereby forming a complex between linaclotide and the first antibody or antigen-binding fragment thereof; and b) assaying for the presence of the complex.

The “complex” formed between linaclotide and the first antibody refers to the molecule formed by the covalent or non-covalent binding between the antibody and linaclotide (e.g. linaclotide-antibody complex). Additional molecules may be chemically bound to linaclotide and the first antibody complex, such as other antibodies, peptides, or detectable labels.

In some embodiments, assays used to detect for the presence of a linaclotide-antibody complex can include, for example, enzyme-linked immunosorbent assay (ELISA), Fluorescence-linked immunosorbent assay (FLISA), electrochemiluminescence (ECL) or other antibody to antibody assay methods.

In other embodiments, the assay includes contacting the linaclotide-antibody complex with a detectable label. In further embodiments, the detectable label is bound to the antibody of the linaclotide-antibody complex. In other embodiments, the detectable label is chemically bound to a second linaclotide molecule. In those embodiments, the anti-linaclotide antibody binds to the second linaclotide molecule which is bound to the detectable label.

In some embodiments, assaying for the presence of the linaclotide-antibody complex comprises the addition of a second antibody to the complex. The second antibody may bind to any portion of the complex. In some embodiments, the second antibody binds to a detectable label. In some embodiments, the second antibody is a horseradish peroxidase (HRP) anti-fluorescein antibody that binds to fluorescein.

C. Methods of Producing Anti-Linaclotide Antibodies

In another aspect of the invention, anti-linaclotide antibodies are produced by: a) conjugating linaclotide to a carrier protein; b) immunizing an animal with the protein conjugated linaclotide to produce an immune response and thereby generating anti-linaclotide antibodies; and c) harvesting the anti-linaclotide antibodies from the immunized animal. In some embodiments, the method of producing anti-linaclotide antibodies further comprises: d) purifying the anti-linaclotide antibodies.

Conjugating linaclotide to a carrier protein may be done using known protein conjugation methods. In some embodiments, protein cross-linking agents such as glutaraldehyde, carbodiimide, succinimide esters, benzidine, periodate, and isothiocyanate are used to conjugate linaclotide to a carrier protein. In some embodiments, the free amine at the N-terminus may be utilized for covalent attachment using glutaraldehyde. In other embodiments, hydrazine is attached at the N-terminus.

A carrier protein is a molecule that may be attached to another molecule to elicit an immune response. A variety of carrier proteins may be conjugated to linaclotide to produce an immune response in an animal. In some embodiments, the carrier protein is a T-cell epitope. In other embodiments, the carrier protein is a homodimer T cell epitope. In other embodiments, the carrier protein is a heterodimer T cell epitope. In other embodiments, the carrier protein is a heterodimer of C5aR agonist/T cell epitope construct. T cell epitope constructs may include constructs from tetanus or ovalbumin. Other carrier proteins may include bovine serum albumin (BSA), or keyhole limpet hemocyanin (KLH). In some embodiments, the carrier protein is a T cell epitope that contain the self-adjuvanting lipid moiety Pam2Cys, a palmitoylated peptide.

Methods of immunizing animals against antigens are known in the art. An immune response refers to the production of antibodies by an animal in response to an antigen. In some embodiments, the animal is immunized by injecting the animal with a solution containing the antigen. In some embodiments, the antigen solution is injected subcutaneously. In other embodiments, the antigen solution is injected intrapertoneally. Any suitable animal may be used to create an immune response by administering the antigen. In some embodiments, the animal is a rabbit. In other embodiments, the animal is a mouse. In other embodiments, the animal is a human.

Anti-linaclotide antibodies may be harvested from the animal following the production of the antibodies from the immune response. Methods of antibody collection are known in the art. For example, the serum or plasma of the animal may be collected. In some embodiments, the antibodies are isolated and purified. In some embodiments, isolation and purification is performed using an affinity column.

In some embodiments, the N-terminus of linaclotide is conjugated to the carrier protein during the production of anti-linaclotide antibodies. The N-terminus of linaclotide refers to the free amine at the terminal Cysₜ. The amine at the N-terminal cysteine may be bound to the carrier protein using methods described above.

In some embodiments, the linaclotide is reduced without disulfide bonds. Reduced linaclotide refers to linaclotide where the three disulfide bonds between cysteine residues are absent and linaclotide is in linear form. Native linaclotide refers to linaclotide where the three disulfide bonds between cysteine residues are present. Linaclotide may be converted from native linaclotide to reduced linaclotide, for example, by reacting native linaclotide with tris(2-caroxyethyl)phosphine (TCEP).

In some embodiments, the carrier protein is conjugated to a dipalmitoyl moiety. dipalmiotyl moieties refer to two palmitic acid molecules attached to a lysine residue in the protein.

In some embodiments, the carrier protein is a T-Cell epitope heterodimer. T cell epitopes refer to the epitopes present on the surface of an antigen-presenting cell. A T cell epitope heterodimer refers to a T cell epitope conjugated to a second protein that is different from the T cell epitope. In some embodiments, the T cell epitope is from canine distemper virus. In other embodiments, the T cell epitope is from influenza A HA protein. In other embodiments, the T cell epitope from OVA class II.

In another embodiment, a method of detecting an antibody or antigen-binding fragment of linaclotide in a biological specimen which comprises a) contacting the specimen with linaclotide or epitope thereof, wherein the linaclotide or epitope thereof is conjugated or bound to a detection label, tag, or substrate, thereby forming a complex between linaclotide and the antibody or antigen-binding fragment thereof of linaclotide and b) assaying for the presence of the complex.

In further embodiments, the method of detecting an antibody or antigen-binding fragment of linaclotide in a biological specimen further comprises performing an assay from the group consisting of dot membrane (dot blot) assay, enzyme-linked immunosorbent assay (ELISA), Fluorescence-linked immunosorbent assay (FLISA) and electrochemiluminescence to detect the presence of the complex. In some embodiments, the method of detecting an antibody or antigen-binding fragment of linaclotide in a biological specimen further comprises conjugating the antibody or antigen-binding fragment thereof of linaclotide with a detection label before contacting the specimen with linaclotide or epitope thereof.

Another aspect of the present invention includes a method for qualifying a manufacturing batch of linaclotide comprising a) providing a batch of linaclotide, b) contacting at least a portion of the batch of linaclotide with anti-linaclotide antibodies or antigen-binding fragment, thereby forming a complex between linaclotide and anti-linaclotide antibodies or antigen-binding fragments thereof, c) quantifying the presence of said complex, and d) correlating the quantity of said complex with a known quantity of complex formed between a reference batch of linaclotide and anti-linaclotide antibodies or antigen-binding fragment thereof. In further embodiments, the anti-linaclotide antibodies or antigen-binding fragment thereof is conjugated to a detectable label to quantify said complex. In some embodiments, the detectable label comprises an enzyme, a radiolabel, a peptide, a linker, a fluorescent molecule, or a chemiluminescent molecule.

In another aspect of the present invention comprises a method of performing a neutralization assay for detecting the degree of inhibition of binding or activity of linaclotide to GC-C, wherein the method comprises contacting linaclotide with anti-linaclotide antibodies or antigen-binding fragment thereof and assaying for the inhibition or activity. In some embodiments, the neutralization assay comprises contacting linaclotide with anti-linaclotide antibodies; incubating cells expressing GC-C with linaclotide and anti-linaclotide antibodies; and detecting the pharmacological binding or activity of linaclotide with anti-linaclotide antibodies.

In further embodiments, methods of neutralizing the activity of linaclotide in a subject is provided comprising administering a therapeutically effective dose of an anti-linaclotide antibody or antigen-binding fragment thereof to a subject, wherein the therapeutically effective dose inhibits the binding of linaclotide to GC-C in said subject.

### EXAMPLES

### Example 1: Generation of Anti-Linaclotide Antibodies

The manufacture of suitable anti-linaclotide antibodies utilized conjugation of linaclotide to carrier proteins via covalent attachment of a mixture of activated KLH and ovalbumin to the N-terminal amine of linaclotide as well as a number of defined structures containing promiscuous T-cell epitope peptides in place of the carrier proteins. In order to avoid side-reactions, the N terminus of linaclotide was modified using 4-FB (formyl benzoic acid). Carrier proteins were modified using activated hydrazine which was then mixed with aldehyde-modified linaclotide.

### Conjugation of Synthetic Peptide to Carrier Proteins

Synthetic peptides are conjugated to carrier proteins prior to immunization of experimental animals. The choice as to the mode of conjugation depends upon the synthetic peptide or carrier protein. Exemplary specific conjugation methods are described below.

In the case of linaclotide, the free amine at the N terminus can be utilized for covalent attachment using glutaraldehyde. Hydrazine may also be added to the N-terminal amine.

Buffers: (PBS (10 mM NaPhosphate, 154 mM NaCl, pH 7.2), Sodium Borate (0.1M borate buffer), 1M Tris Base)
Reagents: (Peptide of interest, Unactivated Carrier protein mix—Glutaraldehyde-mediated conjugation, Activated Carrier protein mix aliquot—MBS or SMCC (for ELISA))

### Procedure—Cysteine-Mediated

1.) Thaw vials of carrier protein in beaker of water
2.) To each 5 mg peptide vial add: a. 1 mL of dH2O b. 5 mg of carrier protein mix—activated with MBS (maleimido-bis-succinimidyl ester)
3.) Place on rotator for 2-3 hours
4.) Bring final volume to 5 mL with PBS

### Procedures—Glutaraldehyde-Mediated

Make the following solutions:

(Solution S1) Peptide+Buffer Solution: In a 1.5 mL microcentrifuge tube, dissolve 2.5-5 mg of peptide in 100 μL of DI water. Add 5004 of borate buffer.

(Solution S2) Carrier Protein+Borate buffer: Make a 10 mg/ml solution of Carrier protein in borate buffer, vortex until protein is in solution

(Solution S3) Glutaraldehyde+Borate buffer

Add 500 μL of solution S2 to solution S1.

Add 100 μL of solution S3 to solution S1 drop wise while vortexing.

Place solution S1 on rocker for 1 hour at RT.

Add 50 μL of 1M Tris to solution S1 tube to stop reaction, vortex. Dilute to 5 ml with PBS.

### Immunization Protocols

The antibody protocol numbers, corresponding peptide sequences, and the immunogen structures utilized were as follows:

### Protocol #1

A construct was prepared by conjugating aldehyde-modified linaclotide prepared using the glutaraldehyde-mediated procedure. The aldehyde-modified linaclotide was conjugated to a peptide heterodimer consisting of the tetanus toxoid promiscuous T-cell epitope and the OVA Class II T-cell epitope. This construct was used to immunize rabbits. The tetanus toxoid promiscuous T-cell epitope and ovalbumin (OVA) Class II T-cell epitope heterodimer had the following amino acid sequence: Ac-QSKNILMQYIKANSKFIGITEL[K-ε-ISQAVHAAHAEINEAGR]G[K-Hz]-amide. Hz=hydrazinoacetic acid conjugated to the epsilon amine of lysine.

### Protocol #2

A construct was prepared by conjugating aldehyde-modified linaclotide prepared using the glutaraldehyde-mediated procedure. The aldehyde-modified linaclotide was conjugated to hydrazine-modified carrier proteins. The hydrazine modified carrier proteins included an equimolar mixture of KLH (keyhole limpet hemocyanin) and ovalbumin. The linaclotide-carrier protein conjugate was used to immunize rabbits. The rabbits produced a robust immune response across multiple HLA alleles.

### Protocol #3-4

Protocols 3-4 utilized a unique structure to elicit immune response independent of carrier proteins and potentially adjuvant. A construct was prepared by conjugating aldehyde-modified linaclotide prepared using the glutaraldehyde-mediated procedure. Aldehyde-modified linaclotide was covalently attached to a synthetic structure consisting of the following elements:

A dipalmitoyl-modified amino acid core, which has been demonstrated to activate TLR2/6 (thus acting as a self-adjuvanting particle)

A promiscuous T-cell epitope (see below for specific epitopes used in protocols 3 and 4).

A furin cleavage site RVKR. Furin is a paired, basic amino acid cleaving enzyme and furin proteolytic sites have been shown to be rapidly cleaved in the endosomal compartment. This results in a movement of released epitopes into the trans-Golgi rather than follow the longer and less efficient route through the endoplasmic reticulum (ER).

Linaclotide—[aldehyde-hydrazine]—furin site—dipalmitoylated core—T-cell epitope

According to the current model, diacylated (in this dipalmitoylated) lipopeptides/lipoproteins induce signaling through TLR2/6. Some diacylated (in this dipalmitoylated) lipopeptides/lipoproteins structures also signal in a TLR6-independent manner. This suggests that both the lipid and peptide part of lipoproteins may take part in the specificity of recognition by TLR2 heterodimers.

### Protocol #3

Hz-GRVKRG[K-SS(K-Palm2)]GGALNNRFQIKGVELKS-OH (T-cell epitope from influenza A HA protein)

The dipalmitoyl moieties are added to the alpha and epsilon amines of the terminal lysine (K).

### Protocol #4

Hz-GRVKRG[K-SS(K-Palm2)]GISQAVHAAHAEINEAGR-OH (T-cell epitope from OVA Class II)

The dipalmitoyl moieties are added to the alpha and epsilon amines of the terminal lysine (K).

### General Immunization Schedule for Protocols 1-4

An AAALAC/USDA/NIH approved farm was used for all animal work (SDIX, Raymond, Me.). Each of the animal protocols utilized 3 rabbits each and the following immunization schedule:

Day 0 Pre-immune serum collected from each rabbit; initial immunization (400 μg immunogen)
Day 14 Boost (200 μg immunogen)
Day 28 Boost (200 μg immunogen)
Day 42 Boost (200 μg immunogen)
Day 52 Production bleed (18-24 ml)
Day 56 Production bleed (18-24 ml)
Day 56 Boost (200 μg immunogen)
Day 66 Production bleed (18-24 ml)
Day 70 Production bleed (18-24 ml)
Day 70 Boost (200 μg immunogen)
Day 80 Production bleed (18-24 ml)
Day 84 Production bleed (18-24 ml)
Day 100 Boost (200 μg immunogen)

### Example 2. Dot Membrane Preparation for Dot Blots

### Dot Membrane Assay Protocol

Preparation of Membrane

Linaclotide was added to membranes for dot blotting in both a reduced and native (disulfide-bonded) form. Linaclotide was exposed to TCEP for disulfide bond reduction which was allowed to evaporate prior to dot blotting. In later analyses, linaclotide was conjugated to BSA (bovine serum albumin) to allow for multiple linaclotide molecules to be exposed on the surface and not bound to the plastic wells.

100 pmol (of what) per 2 μL=1:100 Take 50 μL linaclotide @ 1 mg/mL into 6504, 1× blue bromo/PBS mix.

10 pmol per 2 μL=1:10 Take 100 μL from 1:100 dilution into 900 μL 1× blue bromo/PBS mix.

1 pmol per 2 μL=1:1 Take 100 μL from 1:10 dilution into 900 μL 1× blue bromo/PBS mix.

Rinse membrane in methanol and then soak on rocker in 1×PBS to wet membrane. Briefly wet grid blotting paper and place on saran wrap. Align membrane paper onto the grid blotting paper and hold in place using pins. Use spare blotting paper to remove excess PBS from the membrane by applying pressure.

Use auto pipette to disperse 2 μL drops and dot peptide dilutions as recorded. Let dry at room temperature. Cut membranes as needed and cut off top left corner to determine orientation of peptides. Store dry in −20° C. freezer.

### Assay Conditions:

Wet sample applied membrane in methanol to remove any unbound peptide/protein. Soak in 1×PBS to wet membrane using rocker.

Block non-specific sites by soaking in non-fat dry milk (3 g dry milk per 100 mL 1× TBST, most cases you will need 300 mL of milk for entire procedure). Incubate membranes in milk for 30 minutes.

Set up primary antibody dilutions for incubation: [μg antibody/concentration of antibody]×4 mL milk=μL antibody to add to the 4 mL milk

Put membrane into a ziplock bag with corresponding antibody dilution. Set baggie(s) on rocker and let soak for 1 hour.

Wash three times in 1×TBST for at least 5 minutes each time.

Incubate in secondary antibody for 30 minutes:

2.5 μL secondary Ab per 100 mL milk (if less than 10 membranes)

5.04 secondary Ab per 200 mL milk (if more than 10 membranes)

Wash three times in 1×TBST for at least 5 minutes each time.

Soak membrane in ECL/peroxide mix (MSD) for 1 minute (use 5 μL peroxide in 10 mL ECL, double amounts if more than 10 membranes). Orient the membrane on blotting paper, cover them in saran wrap, secure with scotch tape and place in developer cassette. In dark room, cut top left corner of the film off so orientation can be determined. Start by exposing film to membranes for 1 minute, and increase next film to 5 minutes, 10 minutes, 20 minutes etc. depending on results consecutively.

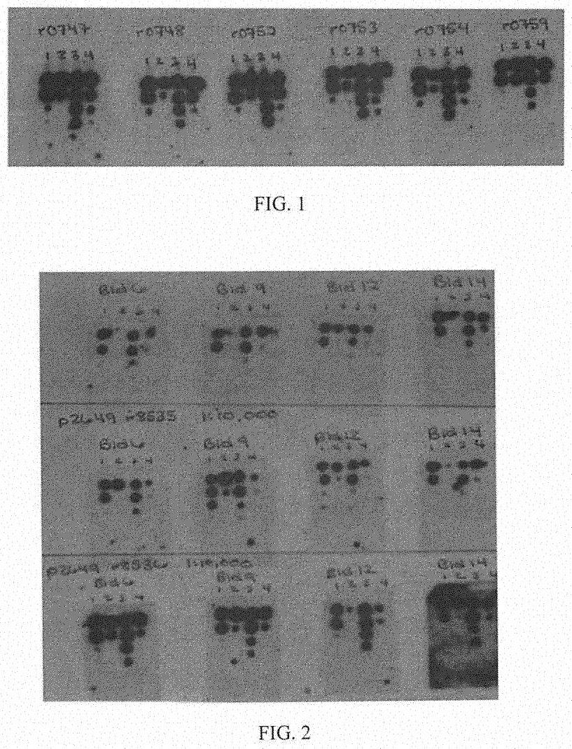

Bleed 2 and bleed 3 from each animal of Protocol 1 was analyzed for linaclotide immunoreactivity by dot blots. FIG. 1 shows the dot blot analysis for bleed 3, with immunoreactivity against both reduced linaclotide (1) and native (disulfide bonded, 2) linaclotide.

### Immunoreactivity of Additional Production Bleeds

Given the success of the initial immunization protocol, rabbits that demonstrated a robust immune response were put on a regular boost and bleed schedule as follows:

### 30 Day Boost and Bleed Schedule

Day 0 Boost (200 μg immunogen in IFA)
Day 10 Production bleed (˜18-24 ml serum)
Day 14 Production bleed (˜18-24 ml serum)

The production bleeds at regular intervals were analyzed by dot blots to determine which bleeds could be used for large scale affinity purification. The dot blots indicated that the immunoreactivity remained robust and in some cases improved with continued boosting and bleeding as shown in FIG. 2.

### Example 3. Antibody Purification Methods

Affinity purification methods were utilized to purify anti-linaclotide antibodies.

Conjugation of linaclotide to affinity resin and the use of this resin as an affinity chromatography media for the purification of anti-linaclotide antibodies.

An initial purification of total IgG was performed using caprylic acid precipitation of non-IgG proteins in an effort to minimize leaching and/or decay of linaclotide affinity column performance.

Use of C-terminally biotinylated linaclotide, SEQ ID NO: 5, for capture on a streptavidin agarose column.

Covalent attachment of linaclotide, SEQ ID NO: 2, or SEQ ID NO: 4 (either via the free N-terminal amine, aldehyde modification, or other linkage) to activated cross-linked agarose beads.

TABLE 1
Peptides used in antibody purification

| Description | Sequence |
|---|---|
| Linaclotide (SEQ ID NO: 1) | C1C2EYC3C1NPAC2TGC3Y |
| SEQ ID NO: 2 | NSSNYC1C2EYC3C1NPAC2TGC3Y |
| SEQ ID NO: 3 | C1C2EYC3C1NPAC2TGC3 |
| SEQ ID NO: 4 | AGSAGSAGSGC1C2EYC3C1NPAC2TGC3Y |
| SEQ ID NO: 5 | C1C2EYC3C1NPAC2TGC3Y -C2-Biotin |

### Immunodepletion Column Purification

AB Special or “Immunodepletion” indicates that a there is a peptide used for affinity purification as well as a particular peptide that has been synthesized to be used only for serum depletion. The serum is run through the immunodepletion column several times to deplete serum of all non-specific antibodies before being incubated with the column specific for the peptide of interest.

### Buffers:

Phosphate buffer—50 mM Na Phosphate, pH 6.5
1 M Tris-Base, pH8.5 (for neutralization)

### 100 mM Glycine, pH 2.3

### Phosphate-Buffered Saline—10 mM NaPhosphate, 154 mM NaCl, pH 7.2

### Materials:

Fritted glass columns, 1×12 cm
200 ml polypropylene bottles
Shaker or rotator

### UV/Vis Spectrophotometer

### Procedure

### Column Prep

Pour gel into a clean column, let any buffer drain out. Use phosphate buffer to rinse conical and ensure all gel gets transferred into the column, let drain.

### Combining Gel and Serum

After appropriate serum has been collected, pour ˜40 mLs into a 50 mL conical.

Pour ˜5 mLs of serum into column containing gel (be sure column tip is secured so no serum drains out)

Pour slurry from column into an appropriate container that will be used for the duration of the incubation.

With the remaining serum in the conical, continue to rinse all slurry out of column by pouring ˜5 mLs at a time into the column, gently rocking by hand and transferring to the incubation container.

Once all gel and serum have been combined together in the incubation container, place on rotator at room temperature for 3 hours, or incubate overnight on a rotator in the cold room.

### Elution

With column secured in clamps and tips removed, position a clean conical to catch serum that drains through.

Pour serum/gel mixture into column. Serum will flow easily at first, slowing as gel accumulates on the bottom. If necessary, us a 30 cc syringe affixed to the cap to push serum through (do not force serum to flow in a steady stream, enough pressure to cause a steady drip is all that is needed).

Once all serum has flowed through the column, it may be necessary to pour some of the drained serum back into the incubation container to be sure any residual gel gets rinsed out and in to the column.

Flow 30 mLs of phosphate buffer through column.

While waiting for phosphate buffer to drain, add Tris-base, pH 8.5 to elution tubes for neutralization (100 μL per ml).

Flow 60 mLs of salt buffer through column.

Flow 10 mLs of phosphate buffer through column (to wash out salt buffer).

Position the 15 mL conicals containing tris base under column so that the one containing the 6 mL mark is ready to collect the flow through first.

Add glycine buffer to the column, collect 6 mLs of glycine flow through in the first conical, and 3 mLs in the remaining 6 conicals.

### Large Scale Affinity Purification Protocols

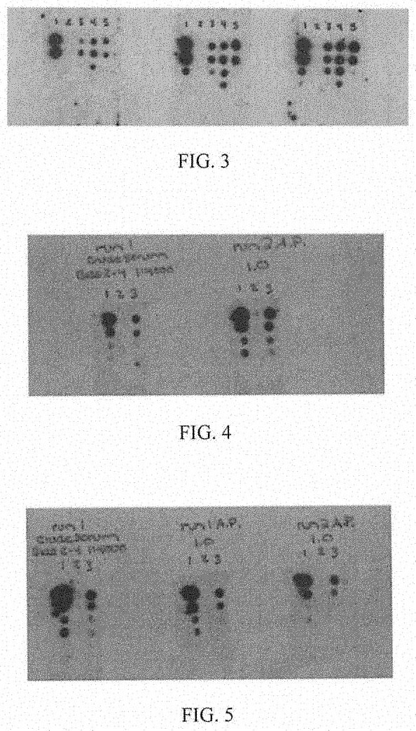

Rabbit sera collected from protocols 1 and 2 were used for large scale affinity purifications. Purification protocols for sera of protocols 1 and 2 consisted of initially 12 rabbits. An initial IgG clean-up step was utilized to enrich the IgG and remove proteases and other serum proteins. Due to the potential problems with other methods for IgG enrichment—ammonium sulfate often causing IgG loss due to aggregation or protein A low pH elution causing issues with immunoreactivity, caprylic acid (CA; octanoic acid) was used to enrich the IgG. CA treatment results in the precipitation of serum proteins other than IgG and is thus the gentlest method for IgG enrichment. An initial test was performed using a comparison of CA enriched IgG for affinity purification versus an affinity purification that used diluted serum only (FIG. 3).

In a first example, purified antibodies from protocol 1 demonstrated a robust immune response to the peptide heterodimer consisting of the tetanus toxoid promiscuous T-cell epitope and the OVA Class II T-cell epitope in 6 of the 12 rabbits, and these animals were boosted and bled to obtain sufficient serum for purification of the antibody requested. See FIG. 1, for dot blot results.

Two ml of serum from bleed 4 from each of the immunoreactive rabbits from Protocol 1 were pooled and analyzed by dot blots as either crude serum or affinity purified antibody as shown in FIG. 4.

In a second example, purified antibodies from protocol 2 demonstrated a robust immune response to the peptide heterodimer consisting of the tetanus toxoid promiscuous T-cell epitope and the OVA Class II T-cell epitope in all 12 rabbits, and these animals were boosted and bled to obtain sufficient serum for purification of the antibody requested. See FIG. 1, for dot blot results.

Two ml of serum from bleed 4 from each of the immunoreactive rabbits from Protocol 2 were pooled and analyzed by dot blots as either crude serum or affinity purified antibody as shown in FIG. 5.

### Results of Various Immunization Affinity Purifications

### Protocol #1

Immunogens: Tetanus toxoid promiscuous T-cell epitope and OVA class II T-Cell epitope linaclotide construct

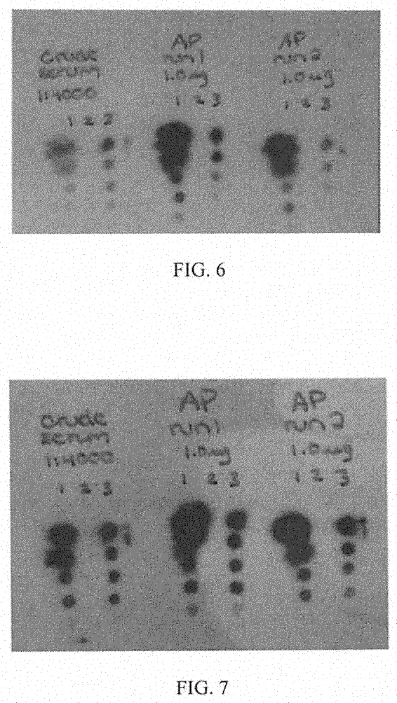

An equimolar mixture of the purified linaclotide-T-cell epitope construct was added to adjuvant (complete Freund's and incomplete Freund's) and the rabbits immunized as described above. FIG. 6 shows all of the immunized rabbits responded as judged by dot blots.

Initial affinity purifications utilized 5 ml of serum pooled from each rabbit, bleed 4. Two separate affinity columns were prepared and one half of the pooled serum was affinity purified utilizing each column.

### Protocol #2

Immunogens: Linaclotide plus KLH and ovalbumin

The sera of protocol 2 was extended through bleed 14 plus a terminal bleed; the sera was evaluated by dot blot as shown in FIG. 7. Initial affinity purifications utilized 5 ml of serum pooled from each rabbit, bleed 4. Two separate affinity columns were prepared and one half of the pooled serum was affinity purified utilizing each column.

### Protocol #3

Immunogens: Hz-GRVKRG[K-SS(K-Palm2)]GGALNNRFQIKGVELKS-OH

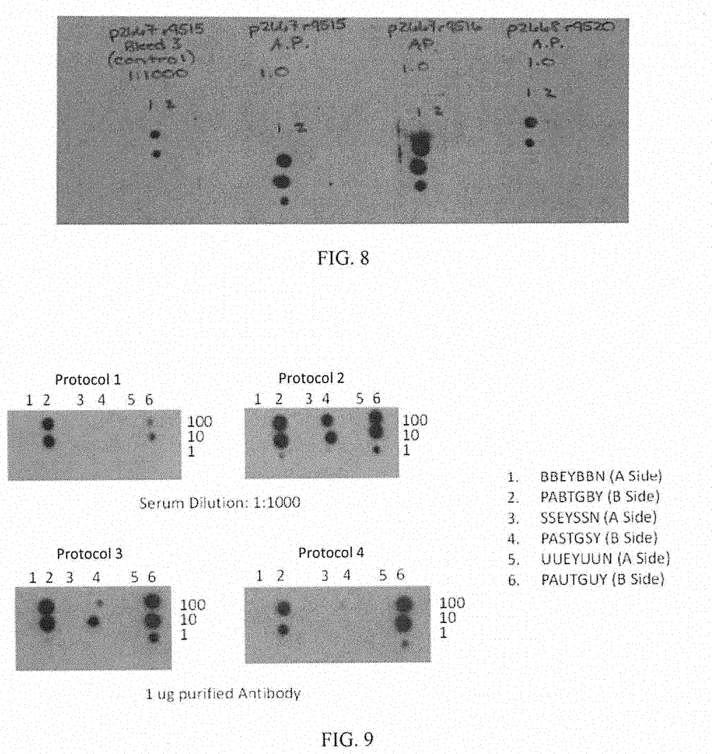

Two of the rabbits immunized using protocol 3 demonstrated immunoreactivity and were continued to be boosted and bled. The AGSA-elongated peptide was used to manufacture affinity columns for Protocols 3 and 4 and 10 ml of serum was affinity purified as an initial test (2.5 ml from each rabbit, bleeds 3 and 4, was pooled). FIG. 8 shows an immune response as determined by dot blot testing.

### Protocol #4

Immunogens: Hz-GRVKRG[K-SS(K-Palm2)]GISQAVHAAHAEINEAGR-OH

One rabbit demonstrated immunoreactivity and this animal was continued to be boosted and bled. A peptide of SEQ ID NO: 8 was used to manufacture affinity columns for Protocols 3 and 4 and 10 ml of serum was affinity purified as an initial test (5 ml of serum from bleeds 3 and 4 was pooled). FIG. 8 shows an immune response as determined by dot blot testing.

### Example 4. Antibody Characterization

All rabbit antisera were tested for binding to linaclotide by dot blot analysis. The antibodies were tested against native linaclotide, reduced linaclotide, and linaclotide bound to bovine serum albumin, as well as 2 forms of linaclotide with additional amino acids on their N terminus (SEQ ID NO: 2 and SEQ ID NO: 4).

Additional analyses were performed to determine the major binding region of the antibodies, either on the N-terminal side or the C-terminal side of linaclotide. Truncated, linear forms of linaclotide were used in dot blots (Table 2). None of the antibodies were able to bind to the N-terminal side of linaclotide (FIG. 9).

### Method of Detection of Anti-Linaclotide Antibodies

1. Dot blots were performed to follow antibody activity during the development of the immune response as well as during processing of the serum. Multiple peptides were utilized on the dot blots including: native linaclotide, reduced linaclotide, BSA-conjugated native linaclotide, and other linaclotide peptide analogs as required (see below for specific details). Other related GC-C agonist peptides were also used including guanylin, uroguanylin and SEQ ID NO: 3.

Dot Blot Analysis of Serum Immunoreactivity from Protocols 1 and 2 Using Peptides Corresponding to Short Regions of Linaclotide

In an effort to determine if antibodies that detect the N-terminal versus C-terminal halves of linaclotide can be isolated, a series of peptides where the cysteines were modified three ways: (1) blocked with acetamidomethyl [Acm]); (2) mutated to serine; or (3) added as Abu instead of cysteine) were manufactured to assess the binding of the anti-linaclotide antibodies from protocols 1 and 2. Based upon the primary sequence of linaclotide, CCEYCCNPACTGCY (SEQ ID NO: 1), the peptide was cut into halves, generating two peptides. The sequences corresponded to CCEYCCN with a C-terminal PEG2-C-amide and PACTGCY-OH with an N-terminal C-PEG2 (Cys used for conjugation to BSA for dot blot analysis).

In both protocols 1 and 2, immunoreactivity was only observed to the C-terminal peptides.

TABLE 2
Truncated linear peptides for binding analysis

| | Sequence | |
|---|---|---|
| Peptide | N-terminal Side | C-Terminal Side |
| Linaclotide | CCEYCCN | PACTGCY |
| Acetamidomethyl Cysteine | BBEYBBN | PABTGBY |
| Serine | SSEYSSN | PASTGSY |
| Aminobutyrate | UUEYUUN | PAUTGUY |

In an effort to further determine the antibody binding site to linaclotide and the specific epitope of linaclotide, peptide motifs (e.g. di-, tri-, and tetra-motifs) of linaclotide may be used for binding studies. The peptides motifs can be developed using any sequential amino acids within the linaclotide sequence. For example, the linear tripeptide and tetrapeptide motifs within linaclotide, as shown in Table 3, may be used.

TABLE 3
Linear Tripeptide and Tetrapeptide Motifs of Linaclotide

| Start Position | Tripeptide | Tetrapeptide |
|---|---|---|
| 1 | CCE | CCEY |
| 2 | CEY | CEYC |
| 3 | EYC | EYCC |
| 4 | YCC | YCCN |
| 5 | CCN | CCNP |
| 6 | CNP | CNPA |
| 7 | NPA | NPAC |
| 8 | PAC | PACT |
| 9 | ACT | ACTG |
| 10 | CTG | CTGC |
| 11 | TGC | TGCY |
| 12 | GCY | |

In some embodiments, the antibodies or antigen-binding fragments as described herein are generated to bind to an epitope of linaclotide. In further embodiments, the antibody or antigen-binding fragment binds to an epitope of linaclotide wherein the epitope comprises the C-terminal tyrosine of linaclotide, the amino acid sequence of Cys Thr Gly Tyr, or dipeptide motif, tripeptide motif, or tetramotif of linaclotide including but not limited to the peptide motifs as described in Tables 2 and 3.

### Example 5. Direct-Binding ELISA

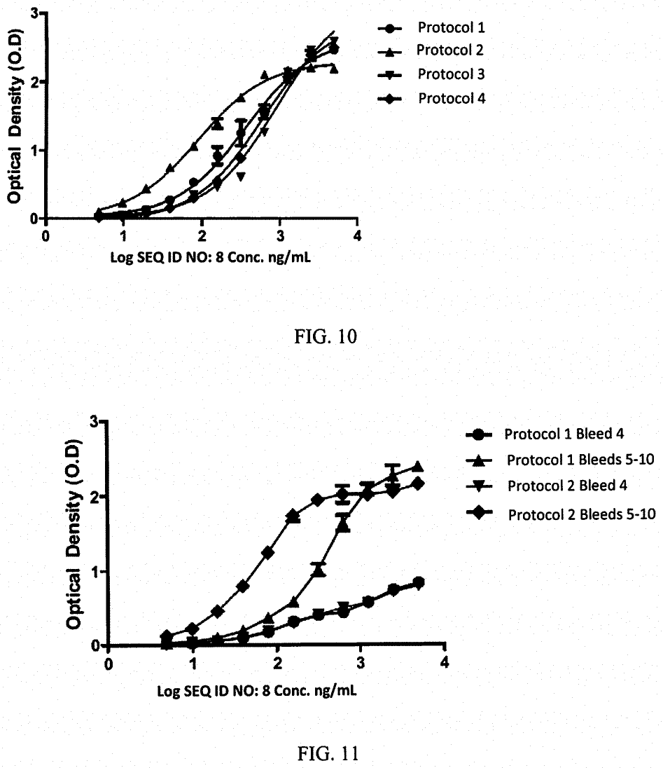

To further characterize the 4 polyclonal anti-linaclotide antibodies, a direct-binding ELISA assay was developed for antibody capture and detection using an antibody concentration of 50 ng/mL. For antibody capture, a streptavidin plate was preincubated for 1-2 hours with a biotinylated form of linaclotide (SEQ ID NO: 8) and washed before anti-linaclotide antibodies were added and incubated overnight at 4° C. For detection of bound antibodies, after the plates were washed, 100 ng/mL goat anti-rabbit horseradish peroxidase (HRP) (Abcam® ab6721) was added and developed according to Abcam protocol provided with Abcam catalog No. ab6721. FIG. 10 presents the binding curves for the 4 polyclonal antibodies: protocol 1 (Bleeds 5 to 10), protocol 2 (Bleeds 5 to 10), protocol 3 (Bleed 4), and protocol 4 (Bleed 4). Earlier and later bleeds for the same protocols were also compared in ELISA; the binding affinities increased with time and continued vaccinations after Bleed 4 (Day 70) (FIG. 11). The concentration of antibodies from protocol 1 for FIG. 11 was 100 ng/mL, and the concentration of antibodies from protocol 2 was 60 ng/mL.

Binding of SEQ ID NO: 9 to the positive control antibody was confirmed in a direct-binding ELISA. Anti-fluorescein antibody conjugated to HRP was used to detect the linaclotide-tracer peptide, SEQ ID NO: 9. The signal increased with increasing antibody concentration.

### Example 6. Bridging Assay

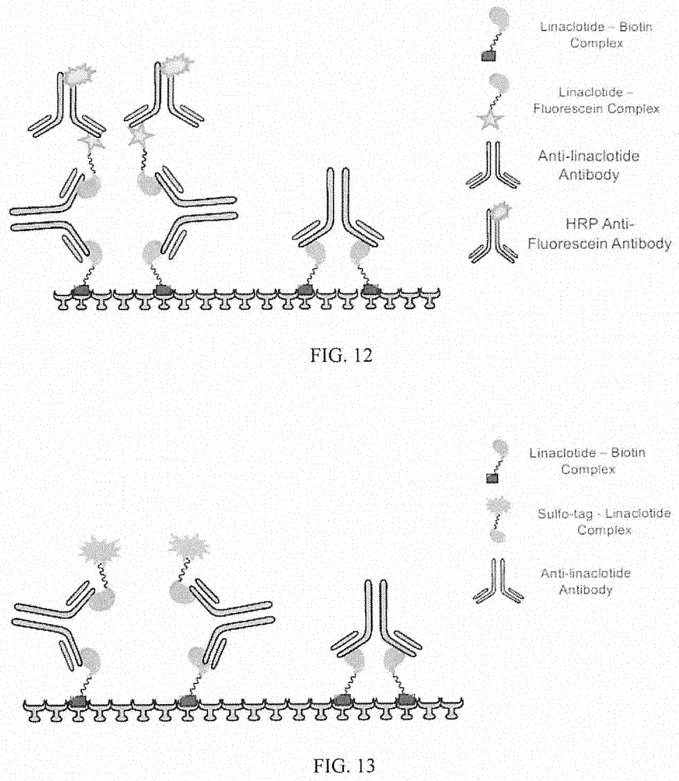

The initial bridging assay was developed using a biotinylated form of linaclotide, SEQ ID NO: 8, for antibody capture, and the linaclotide-tracer peptide, SEQ ID NO: 9, for detection on a streptavidin plate. Simultaneous binding of both the capture and detection peptides resulted in a positive signal for anti-linaclotide antibodies (FIG. 12). A sheep anti-fluorescein antibody conjugated to HRP was used for detection of SEQ ID NO: 9 to enhance the signal over direct detection of the fluorescein moiety by ultraviolet-visible spectroscopy (UV/Vis). In this initial assay, the capture peptide (biotin-linaclotide), detection peptide (fluorescein-linaclotide), and anti-linaclotide antibodies were first incubated overnight in solution to allow complexes to form. These complexes were captured on a streptavidin plate, which was then washed. Bound complexes were detected with an HRP-conjugated anti-fluorescein antibody followed by addition of the HRP substrate and colorimetric detection.

Bridging Assay with MSD-Sulfo-Tag Tracer

The Bridging Assay was adapted from Meso-Scale Discovery (MSD) manufacturer instructions for using the MSD platform. For detection of anti-linaclotide antibodies, the MSD Sulfo-Tag label was attached to the N terminus of an elongated form of linaclotide (SEQ ID NO: 4) to produce a detection peptide (SEQ ID NO: 11). The procedure for attaching SULFO-TAG to peptides is provided in MSD's manufacturer protocol. In addition to this detection peptide, the MSD assay method used SEQ ID NO: 8 (biotinylated linaclotide) for antibody capture on MSD's High Bind Avidin Gold Plate. In this assay, the capture peptide (biotin-linaclotide), detection peptide (Sulfo-Tag-labeled linaclotide), and anti-linaclotide antibodies are first incubated overnight in solution to allow complexes to form. The complexes are captured on the MSD's avidin plate, which is then washed. Bound complexes are detected using ECL technology (FIG. 13). This assay is independent of antibody isotype and detects IgG, IgA, and IgM.

### Initial Assay Testing

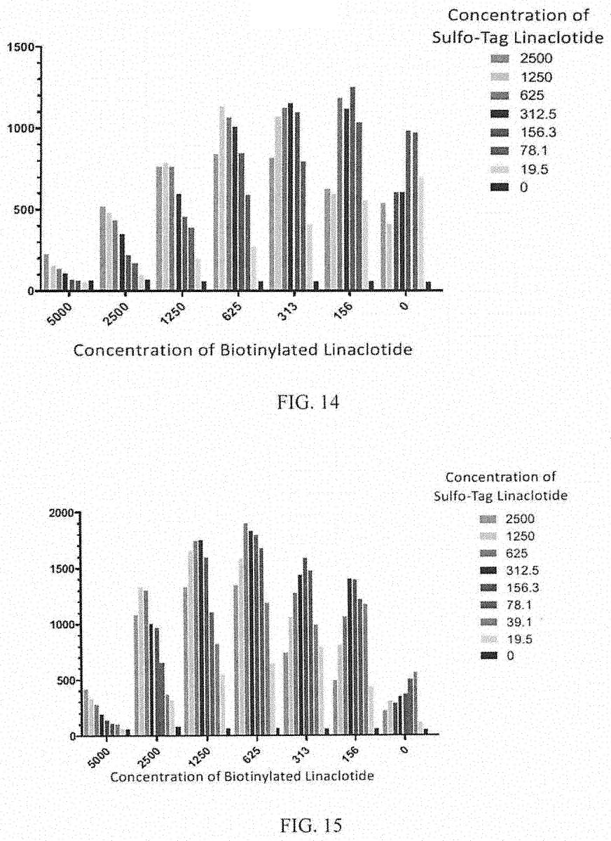

Initial concentrations of the capture peptide (SEQ ID NO: 8) and the detection peptide (SEQ ID NO: 11) were determined in checkerboard titrations in which dilutions of each peptide were tested against each other using 2 μg/mL of sera produced from protocol 1 (Bleed 4) or protocol 2 (Bleed 4). Results of the checkerboard titrations are shown in FIG. 14 and FIG. 15 for sera from protocol 1 and protocol 2, respectively, using an antibody concentration of 2 ng/mL.

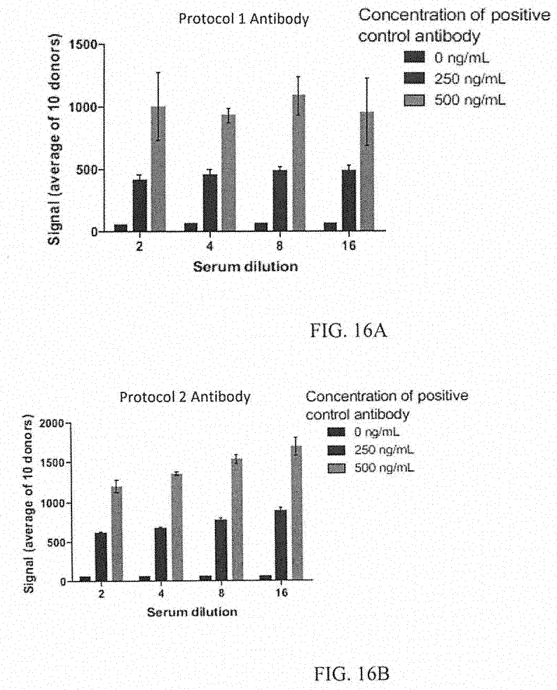

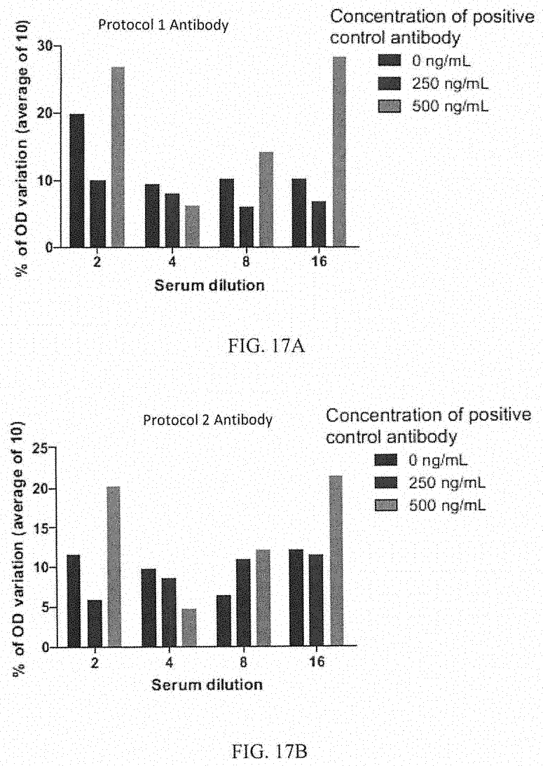

The assay was tested using 0, 250, and 500 ng/mL of positive control antibody (sera from protocol 1 and 2) spiked into 10 individual human donor serum samples. On average, the signal for both positive control antibodies at 250 ng/mL was at least 6 times greater than the no-antibody control. The signals for the 500 ng/mL antibody assays were approximately twice that of the 250 ng/mL assays, indicating that the responses are dependent on antibody concentration and are consistent for serum dilutions up to 1:16. FIGS. 16A and 16B show the effect of serum dilution on the bridging assay signals using 100 μL of 625 ng/mL SEQ ID NO: 8, and 100 μL of 625 ng/mL SEQ ID NO: 11. Variability among the individuals was below 20% for serum dilutions of 1:4 and 1:8. Serum dilutions of 1:2 and 1:16 had higher variability; therefore, serum dilutions of 1:4 or 1:8 were determined as optimal for validation. FIGS. 17A and 17B show the percent variability among individual serum sample signals using 100 μL of 625 ng/mL SEQ ID NO: 8, and 100 μL of 625 ng/mL SEQ ID NO: 11.

### Example 7. Confirmatory Assay

The confirmatory assay was developed as a variation of the ELISA-based bridging assay (Example 6) with competition using fluorescein for detection. The peptide antibody incubation mixture was spiked with native linaclotide, and the concentrations of positive control antibody, capture peptide, and detection peptide were kept constant.

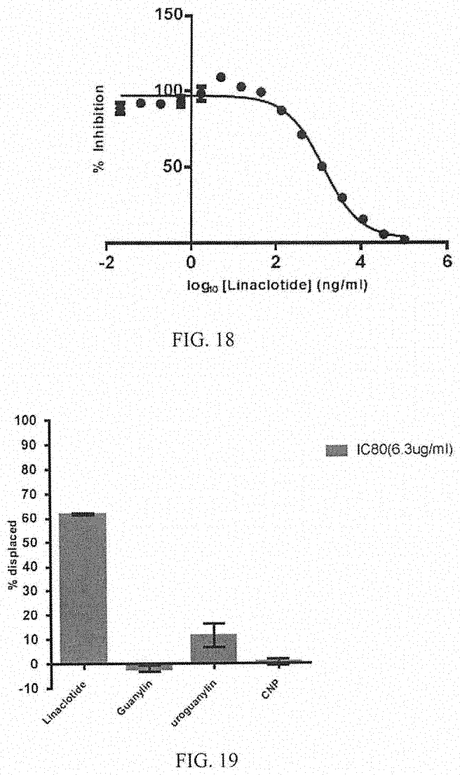

The addition of increasing amounts of linaclotide to the peptide-antibody mixture inhibited binding of the capture and/or detection peptide to the antibody. A binding curve was generated using Prism® v. 5.01 (GraphPad Software), and data were analyzed with non-linear regression to calculate the 50% and 80% inhibitory concentration (IC₅₀ and IC₈₀, respectively) values of 1.4 and 6.3 μg/mL, respectively. FIG. 18 shows the binding curve using 500 ng/mL antibody obtained from protocol 2, 300 ng/mL SEQ ID NO: 8, and 312 ng/mL SEQ ID NO: 9. This demonstration of competitive inhibition confirmed the binding of linaclotide to the positive control antibodies that were generated by Protocol 2.

As described above, the confirmatory assay was developed using increasing concentrations of linaclotide to compete with the capture and detection peptides. The confirmatory assay was validated using a concentration of 20 μg/mL linaclotide for competition. Due to the high concentration of linaclotide required to compete away the assay signal, the low positive control (LPC) of 44 ng/mL that was established for the screening assay could not be validated for the confirmatory assay; therefore, increasing LPC concentrations up to the high positive control (HPC) of 500 ng/mL were tested. Ultimately, a concentration of 150 ng/mL was established as a suitable LPC for the confirmatory assay. Consequently, the combined assay sensitivity of the anti-linaclotide screening and confirmatory assays is 150 ng/mL antibody.

### Example 8. Cross-Reactivity Assay

The cross-reactivity assay to test for cross-reactivity to the endogenous hormones, uroguanylin and guanylin, is a competition assay similar to the confirmatory assay (Example 7).

Competition with Uroguanylin and Guanylin

To determine if the positive control antibody produced by protocol 2 could bind either of the endogenous hormones, uroguanylin or guanylin peptides were used in place of linaclotide in a competition assay similar to the confirmatory assay described in Example 7. During development, the IC80 concentration for linaclotide, 6.3 μg/mL, was chosen as a relevant test concentration for cross-reactivity because it is on the higher end of the linear range of the inhibition curve (FIG. 18). This concentration (6.3 μg/mL) is approximately 10,000 times the systemic circulating levels of uroguanylin and guanylin. A non-related peptide, C-type natriuretic peptide (CNP), was added as a negative control in this assay. Neither uroguanylin nor guanylin was able to inhibit the binding of the detection peptides to anti-linaclotide antibodies more than the negative control peptide, CNP. FIG. 19 shows the cross-reactivity of anti-linaclotide antibodies obtained using protocol 2 using 500 ng/mL of antibody obtained using protocol 2 and 6.3 μg/mL of the tested peptides. The addition of 6.3 μg/mL of linaclotide (positive control for this assay) inhibited binding by approximately 60% under the same conditions described in Example 7. These results indicate that, under these experimental conditions, uroguanylin and guanylin are not recognized by antibodies produced by protocol 2 (FIG. 19). However, these results could be limited by the antibody binding affinities of uroguanylin and guanylin versus that of linaclotide.

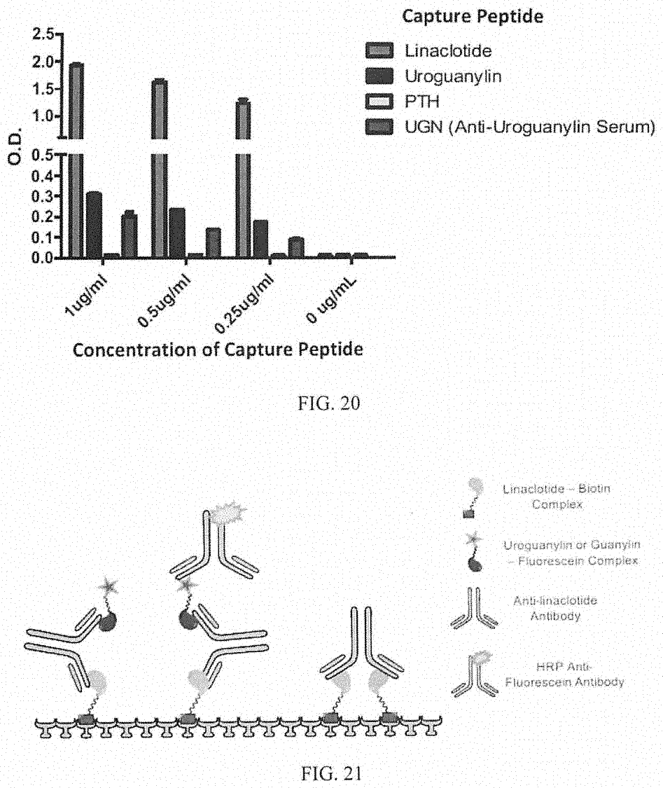

To further characterize the interactions between the positive control antibody and the cross-reacting peptides, a direct-binding ELISA experiment was conducted in which varying concentrations of biotinylated peptides (linaclotide, uroguanylin, and negative control parathyroid hormone [PTH]) were immobilized on the plate for antibody capture. The assay was performed using either sera from protocol 1 or anti-uroguanylin serum (Abcam ab52806) in ELISA buffer; no adequate control antibody to guanylin is available. HRP-anti-rabbit secondary antibody was used for the detection of anti-linaclotide antibodies, and HRP-anti-mouse secondary antibody was used for the detection of anti-uroguanylin antibodies from the antiserum. FIG. 20 indicated that antibody produced by protocol 1 binds to uroguanylin in a concentration-dependent manner using 60 ng/mL of antibody of protocol 1, a ratio of antiserum to uroguanylin at a 1:50 dilution and secondary horseradish peroxidase antibodies at a concentration of 100 ng/mL (PTH is parathyroid hormone). The signal for uroguanylin binding was approximately 15% of the signal obtained for linaclotide under the same conditions.

### Bridging Assay to Assess Binding of Uroguanylin or Guanylin Peptides to the Positive Control Antibodies Produced by Protocol 2

TABLE 4
Peptides Synthesized for Assay Development

| Molecule Description | Sequence |
|---|---|
| Linaclotide (SEQ ID | C1 C2 EYC3 C1 NPAC2 TGC3 Y |
| NO: 1) | |
| SEQ ID NO: 3 | C1 C2 EYC3 C1 NPAC2 TGC3 |
| (Guanylin) | PGTC1 EIC2 AYAAC1 TGC2 |
| (Uroguanylin) | NNDC1 ELC2 VAVAC1 TGC2 L |
| SEQ ID NO: 6 | Biotin-C1 C2 EYC3 C1 NPAC2 TGC3 Y |
| SEQ ID NO: 7 | Biotin-NSSNY C1 C2 EYC3 C1 NPAC2 TGC3 Y |
| SEQ ID NO: 4 | AGSAGSAGSG C1 C2 EYC3 C1 NPAC2 TGC3 Y |
| SEQ ID NO: 8 | Biotin-AGSAGSAGSG C1 C2 EYC3 C1 NPAC2 TGC3 Y |
| SEQ ID NO: 5 | C1 C2 EYC3 C1 NPAC2 TGC3 Y -C2-Biotin |
| SEQ ID NO: 9 | Fluorescein-C1 C2 EYC3 C1 NPAC2 TGC3 Y |
| SEQ ID NO: 10 | Fluorescein-NNDC1 ELC2 VAVAC1 TGC2 L |
| SEQ ID NO: 11 | Sulfo-Tag-AGSAGSAGSG C1 C2 EYC3 C1 NPAC2 TGC3 Y |
| SEQ ID NO: 12 | Fluorescein-PGTC1 EIC2 AYAAC1 TGC2 |

C1: Disulfide bond #1
C2: Disulfide bond #2
C3: Disulfide bond #3

A bridging cross-reactivity assay similar to the screening assay (Example 6) was set up to detect direct binding of the positive control antibody to both linaclotide and uroguanylin or guanylin. For use as detection peptides, uroguanylin and guanylin were synthesized with fluorescein coupled to their N terminus (SEQ ID NO: 10 and SEQ ID NO: 12, respectively; Table 4). This cross-reactivity assay used SEQ ID NO: 8 (Table 4) as the capture peptide, just as in the screening assay, and either SEQ ID NO: 10 (for uroguanylin) or SEQ ID NO: 12 (for guanylin) as the detection peptide. Sheep anti-fluorescein HRP-conjugated antibody was used for detection of positive control antibodies. The antibody must directly bind both the linaclotide-capture peptide and uroguanylin or guanylin to generate a signal in the assay (FIG. 21). A fluorescein-modified PGC1a peptide was purchased for use as a negative control for cross-reactivity.

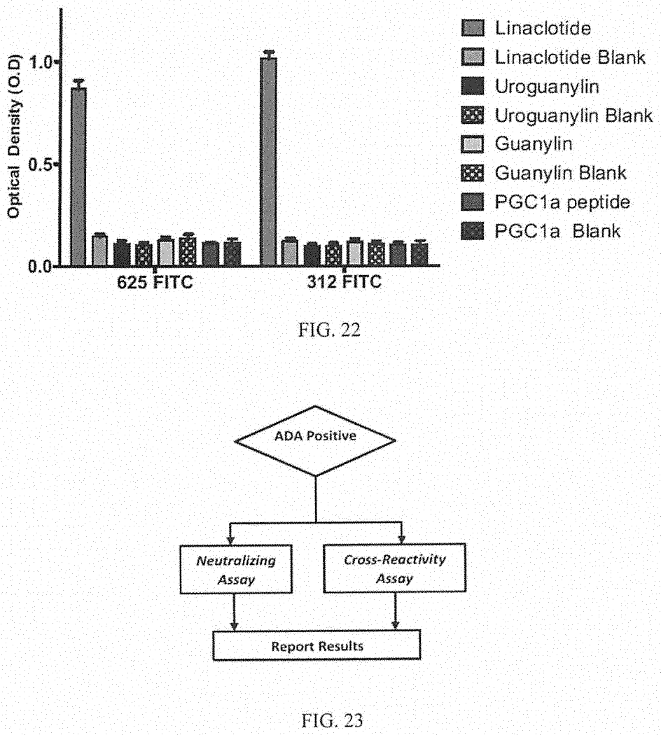

This cross-reactivity assay was performed using 500 ng/mL of positive control antibody produced by protocol 1, 312 ng/mL of SEQ ID NO: 8, and 2 concentrations (312 and 625 ng/mL) of fluorescein-modified peptides for each set of reactions. Only linaclotide was able to generate a positive signal in the direct-binding assay for cross-reactivity (FIG. 22). Positive control antibody is excluded from blank incubations.

As in the competition study, the bridging assay for cross-reactivity could be limited by the antibody binding affinities of uroguanylin and guanylin versus that of linaclotide. The modified form of linaclotide, which is included in each reaction for antibody capture, may compete for all of the binding sites on the antibody, which in turn may not allow a detection peptide to bind. Suitable positive control antibodies to guanylin and uroguanylin are not available for this bridging assay.

### Example 9. Electrochemiluminescence Assay

### Experimental Design

The electrochemiluminescence assay was used as a validation of a method to detect anti-linaclotide antibodies in human serum using a MSD electrochemiluminescence protocol. Low positive control (LPC), high positive control (HPC), relative light units (RLU), coefficient of variation (CV) and confirmation cut points were determined using MSD procedures. The positive control antibodies used were the antibodies harvested from protocol 2.

The assay is an immunogenicity bridging-based assay developed on MesoScale Discovery Electrochemiluminescence (ECL) technology. It is performed by first mixing a biotinylated-linaclotide analog, a sulfotag-labeled linaclotide analog, and the clinical sample together in a multi-well plate. The presence of anti-linaclotide antibodies in the clinical sample forms an immune complex with a biotin-labeled linaclotide analog and a sulfotag-labeled linaclotide analog. The immune complex is subsequently captured on a streptavidin coated MesoScale Discovery multi-well plate followed by a wash step. The captured immune complex is then detected by electrochemiluminescence generated by the sulfotag-labeled linaclotide portion of the immune complex. The signal generated by the sample is then compared to a pre-determined screening cut point to determine if the sample is positive for the presence of anti-linaclotide antibodies. Samples that are determined to be positive are subjected to a second immunogenicity confirmation assay which is performed by mixing a biotinylated-linaclotide analog, a sulfotag-labeled linaclotide analog, the clinical sample, and unlabeled linaclotide together in a multi-well plate. The immune complex is subsequently captured on a streptavidin-coated MesoScale Discovery multi-well plate followed by a wash step. The captured immune complex is then detected by electrochemiluminescence generated by the sulfotag-labeled linaclotide portion of the immune complex. The signal generated by the sample is then compared to a pre-determined confirmation assay cut point to determine if the sample is positive for the presence of anti-linaclotide antibodies. Samples that are confirmed positive by this assay are subsequently tested at different dilutions to determine the titer of the anti-linaclotide antibody.

A general list of the equipment and critical reagents used in this method validation follows.

### Reagents

Polyclonal Rabbit anti-Linaclotide Antibody(900 μg/mL) positive control

### Linaclotide (5.0 mg)

### Uroguanylin (5.0 mg)

### Guanylin (1.0 mg)

### Biotin-Conjugated Linaclotide

### SulfoTag-Conjugated Linaclotide

### Equipment

| | |
|---|---|
| Equipment | Model and Supplier |
| Instrument | MSD Sector Imager 2400 Electrochemiluminescent Reader |
| Microplate Washer | Biotek ELx405 |
| Microtiter Plate Shaker | Titertek |
| pH Meter | Mettler Toledo - SevenEasy (or equivalent) |
| Analytical Balance | Mettler Toledo XP26 (or equivalent) |
| Ultralow Temp Freezer | Thermo Scientific REVCO - Ultima PLUS (or equivalent) |

### Data Handling

The endpoint data were collected using a Sector Imager 2400 electrochemiluminescent reader. The relative fluorescence data were processed in StatLIA, version 3.2. Replicate readings of each control or sample were averaged and shown in the tables as mean values. Values reported as Positive/Negative ratios were calculated by dividing the individual positive RLU value by the mean of the NC values from the value's respective plate. All log values are in base 10. Statistics shown in the tables were calculated in MS Excel and/or JMP.

### Selectivity

The selectivity of the assay was assessed by spiking five male and five female individual human serum samples with positive control anti-linaclotide antibody obtained by protocol 2 at the HPC (500 ng/mL) and screening LPC (44 ng/mL) levels. The experiment was performed in six different runs. The results of the selectivity experiment are provided in Table 5. The results of the experiment described herein indicate acceptable selectivity in the assay.

### Specificity

Specificity (cross-reactivity) of the assay was assessed by spiking ten drug-naive individual human serum samples containing rabbit anti-linaclotide antibody at the HPC (500 ng/mL) and the screening LPC (44 ng/mL) levels with 10 μg/mL Uroguanylin or Guanylin. At the same time, ten replicates of the antibody-spiked samples (without Uroguanylin or Guanylin) at each level were included to establish the % Inhibition. The following formula was used to calculate the % Inhibition for each individual:

% Inhibition=[(1−(Drug with Antibody/Antibody alone)]*100

The specificity data are located in Table 6 for Uroguanylin, and Table 7 for Guanylin. All inhibition values were <20% for each of the individuals.

TABLE 5
Selectivity of Rabbit Positive Control Antibody in Human Serum
Neat Unspiked and Spiked at the HPC (500 ng/mL)

| | | | | | | Mean | |
|---|---|---|---|---|---|---|---|
| Sample | | RLU | CV | | CV | HPC | % |
| No. | Gender | (neat) | (%) | RLU (HPC) | (%) | response | Difference |
| 1 | M | 45 | 3.1 | 341 | −1.2 | 315^{b} | 8.3 |
| 2 | M | 41 | 5.2 | 326 | −3.3 | | 3.3 |
| 3 | F | 45 | 0.0 | 285 | −1.5 | | −9.5 |
| 4 | F | 50 | 12.9 | 318 | −3.1 | | 1.0 |
| 5 | F | 56 | 6.4 | 329 | −4.1 | | 4.3 |
| 6 | F | 40 | 5.4 | 331 | −5.6 | | 5.1 |
| 7 | M | 41 | 8.7 | 363 | −3.5 | | 15.2 |
| 8 | F | 42 | 5.1 | 336 | −3.4 | | 6.7 |
| 9 | M | 43 | 6.6 | 332 | −6.4 | | 5.4 |
| 10 | M | 39 | 14.5 | 307 | −1.8 | | −2.5 |

Spiked at 44 ng/mL (Screening LPC)

| Sample | | RLU | CV | Mean LPC | |
|---|---|---|---|---|---|
| No. | Gender | (LPC) | (%) | response | % Difference |
| 1 | M | 66 | 9.7 | 63^{b} | 4 |
| 2 | M | 68 | 6.2 | | 7.9 |
| 3 | F | 60 | 0.0 | | −4.8 |
| 4 | F | 61 | 0.0 | | −3.2 |
| 5 | F | 79 | 5.4 | | 25.4^{c} |
| 6 | F | 72 | 1.0 | | 13.5 |
| 7 | M | 62 | 2.3 | | −1.6 |
| 8 | F | 68 | 5.2 | | 7.1 |
| 9 | M | 66 | 2.1 | | 4.8 |
| 10 | M | 71 | 10.0 | | 12.7 |

^{a}% Difference = ((Mean RLU − _{MeanHPC or LCP Response})/Mean_{HPC or LPC Response})* 100
^{b}Mean HPC or LPC response
^{c}Value > 20%

TABLE 6
Specificity in Samples Spiked with Anti-Linaclotide Antibody at
the HPC and LPC Levels Pre-Incubated with and without Uroguanylin

| | | | | | | |
|---|---|---|---|---|---|---|
| | | | | Antibody and | | |
| Sample | Confirmation | Antibody | | 10 μg/mL | | Percent |
| No. | Cut point | alone | CV (%) | Uroguanylin | CV (%) | Inhibition^{a} |

Anti-Linaclotide Antibody at the HPC (500 ng/mL)

| | | | | | | |
|---|---|---|---|---|---|---|
| 1 | 25 | 160 | 14.3 | 157 | 11.7 | 1.9 |
| 2 | | 165 | 15.8 | 147 | 15.5 | 10.7 |
| 3 | | 185 | 8.8 | 151 | 11.6 | 18.6 |
| 4 | | 160 | 11.2 | 145 | 0.9 | 9.5 |
| 5 | | 147 | 10.0 | 164 | 4.6 | −11.3 |
| 6 | | 180 | 2.5 | 162 | 9.4 | 10.1 |
| 7 | | 166 | 14.8 | 185 | 16.1 | −11.7 |
| 8 | | 178 | 16.1 | 180 | 10.6 | −1.3 |
| 9 | | 168 | 11.4 | 169 | 5.3 | −0.5 |
| 10 | | 181 | 5.1 | 180 | 6.5 | 0.6 |

Anti-Linaclotide Antibody at the Screening LPC (44.0 ng/mL)

| | | | | | | |
|---|---|---|---|---|---|---|
| 1 | 25 | 55 | 16.7 | 59 | 10.8 | −6.6 |
| 2 | | 47 | 11.1 | 39 | 12.9 | 18.4 |
| 3 | | 52 | 4.5 | 44 | 3.2 | 15.7 |
| 4 | | 54 | 7.0 | 52 | 6.1 | 4.8 |
| 5 | | 49 | 13.3 | 51 | 2.8 | −3.8 |
| 6 | | 52 | 17.3 | 52 | 18.3 | −1.5 |
| 7 | | 54 | 8.8 | 52 | 19.6 | 3.2 |
| 8 | | 62 | 4.1 | 59 | 3.6 | 5.1 |
| 9 | | 52 | 9.3 | 52 | 0.7 | −0.5 |
| 10 | | 51 | 3.7 | 53 | 0.7 | −4.1 |

^{a}% Inhibition = [1-(Antibody + Uroguanylin/Antibody Alone)]*100

TABLE 7
Specificity in Samples Spiked with Anti-Linaclotide Antibody at
the HPC and LPC Levels Pre-Incubated with and without Guanylin

| | | | | | | |
|---|---|---|---|---|---|---|
| | | | | Antibody and | | |
| Sample | Confirmation | Antibody | | 10 μg/mL | | Percent |
| No. | Cut point | alone | CV (%) | Uroguanylin | CV (%) | Inhibition^{a} |

Anti-Linaclotide Antibody at the HPC (500 ng/mL)

| | | | | | | |
|---|---|---|---|---|---|---|
| 1 | 19.1 | 228 | 6.7 | 239 | 10.9 | −4.5 |
| 2 | | 225 | 14.9 | 225 | 16.5 | 0.0 |
| 3 | | 205 | 11.9 | 199 | 13.0 | 3.2 |
| 4 | | 193 | 13.0 | 179 | 14.7 | 7.6 |
| 5 | | 171 | 15.8 | 177 | 16.5 | −3.4 |
| 6 | | 178 | 15.8 | 174 | 15.6 | 2.2 |
| 7 | | 168 | 18.5 | 181 | 15.5 | −7.8 |
| 8 | | 150 | 8.4 | 155 | 12.8 | −3.9 |
| 9 | | 133 | 13.5 | 135 | 15.5 | −1.9 |
| 10 | | 121 | 10.9 | 130 | 14.5 | −7.1 |

Anti-Linaclotide Antibody at the Screening LPC (44.0 ng/mL)

| | | | | | | |
|---|---|---|---|---|---|---|
| 1 | 19.1 | 66 | 9.9 | 71 | 7.0 | −7.1 |
| 2 | | 62 | 11.7 | 57 | 6.9 | 8.2 |
| 3 | | 57 | 1.3 | 55 | 7.1 | 3.4 |
| 4 | | 62 | 4.0 | 54 | 9.8 | 12.5 |
| 5 | | 60 | 4.3 | 53 | 3.3 | 11.0 |
| 6 | | 60 | 7.6 | 55 | 5.8 | 7.4 |
| 7 | | 56 | 2.9 | 53 | 14.1 | 6.4 |
| 8 | | 65 | 2.9 | 63 | 9.1 | 3.6 |
| 9 | | 57 | 6.4 | 57 | 4.3 | 0.1 |
| 10 | | 55 | 3.7 | 51 | 0.0 | 7.0 |

^{a}% Inhibition = [1-(Antibody + Uroguanylin/Antibody Alone)]*100

### Example 10. Neutralization Assay

Patient serum samples that are confirmed positive for anti-linaclotide antibodies were tested for neutralization activity (FIG. 23).

The neutralizing assay is a cell-based bioassay built on linaclotide's mechanism of action; consequently, any interference in that mechanism by potential neutralizing antibodies would be detected. A critical aspect of neutralization assay development is the identification of a suitable positive control, i.e., a control demonstrating that the assay is detecting neutralizing activity. Eight candidates were used as positive controls: 4 polyclonal anti-linaclotide antibodies (produced using protocols 1, 2, 3, 4), antibodies to GC-C(3 sources), and a chimeric molecule containing the ligand-binding domain of GC-C fused to human IgG Fc.

### T84 Cell-Based Assay

### Overview

The neutralizing assay is a cell-based assay built on a method used for the in vitro pharmacological characterization of linaclotide. This method assesses linaclotide's pharmacological activity in cultured human colon carcinoma T84 cells, which express GC-C, the target of linaclotide. Binding of linaclotide to GC-C results in a correlated increase in cGMP concentration in the T84 cells. Following lysis of the T84 cells, this intracellular cGMP may be measured using LC-MS, in a validated assay. The amount of cGMP accumulated in the T84 cells correlates with the concentration of linaclotide that was incubated with the cells. For the neutralization assay, anti-linaclotide antibodies with a neutralizing effect would result in a decreased intracellular cGMP level compared with the cGMP level from a linaclotide-only control.

In this assay, 2×10⁵ T84 cells are seeded overnight in a 96-well plate. The media is then removed, and the T84 cells are pre-incubated with cell culture medium (Dulbecco's modified Eagle medium, DMEM) containing the general phosphodiesterase (PDE) inhibitor, 3-isobutyl-1-methylxanthine (IBMX). Linaclotide concentrations ranging from 0.1 to 10,000 nM are added, and the cells are incubated for 30 minutes at 37° C. Following incubation and removal of media, the cells are lysed in 0.1 M HCl, cell debris is removed by centrifugation, and the pH of the supernatant-containing cGMP is neutralized. The concentration of cGMP is determined by LC-MS using [¹³C¹⁵N-cGMP] as internal standard.

### Concentration Response Curves in T84 Cells in the Presence of Cell Culture Media and Serum

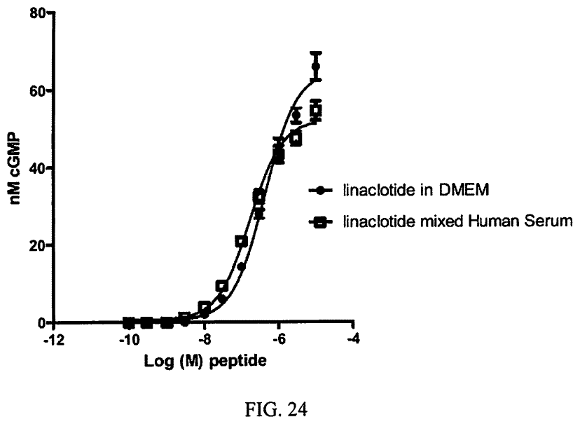

This assay works when the T84 cells are incubated with cell media (DMEM) containing linaclotide as well as when the T84 cells are incubated with human serum containing linaclotide. Representative concentration-response curves for both DMEM and human serum are presented in FIG. 24. The overlap of the two curves demonstrates the equivalence of the assay in either medium. The ability to conduct this assay in the presence of serum indicates that the assay is not sensitive to matrix effects and makes it ideal for detecting neutralizing antibodies. Based on these concentration-response curves in FIG. 24, 100 nM linaclotide was chosen for the neutralization experiments since this concentration falls on the linear portion of the curve where the cGMP accumulation activity is expected to be sensitive to neutralization by antibodies.

### Positive Controls for Neutralization Assay

### Assay Results Using Polyclonal Antibodies as Positive Controls

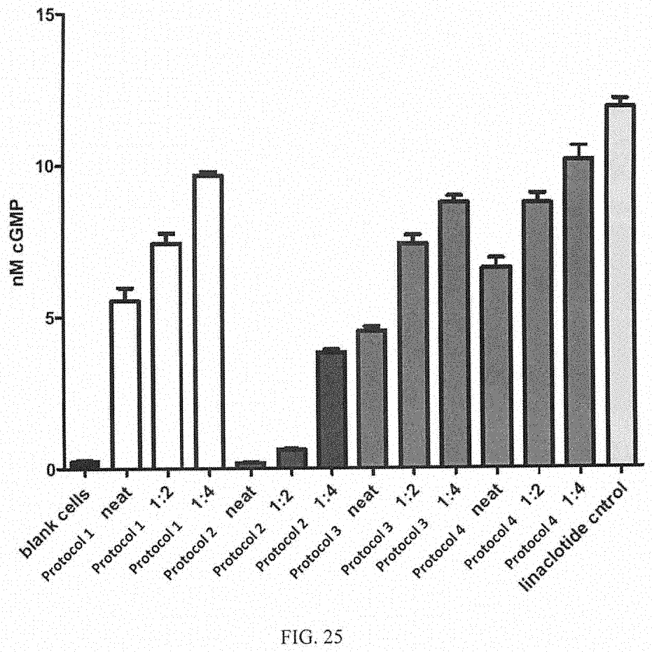

The T84 bioassay described above was used to determine if the polyclonal anti-linaclotide antibodies developed under protocols 1, 2, 3, and 4 inhibited the intrinsic binding of linaclotide to GC-C thereby resulting in a decrease in cGMP concentration in T84 cells. Initial neutralization experiments using anti-linaclotide antibodies produced under protocols 1 and 2 (Bleed 2) showed no neutralization of the pharmacological activity of linaclotide at GC-C. However, in later neutralization experiments anti-linaclotide antibodies produced using protocols 1 and 2 (Bleeds 5 to 10), and antibodies produced using protocols 3 and 4 (Bleed 4) did show neutralization of the pharmacological activity of linaclotide at GC-C(FIG. 25). In this test, linaclotide at 100 nM, which is within the linear range of the concentration-response curve (FIG. 24), was preincubated with purified anti-linaclotide antibody at 3 concentrations (3,200 nM [neat], 1600 nM [1:2], and 800 nM [1:4]) for 3 hours to allow binding of the antibody to linaclotide. These concentrations represent molar ratios of antibody to linaclotide of 32/1, 16/1, and 8/1, respectively. These mixtures were then added to the T84 cells and incubated as described above. The results indicated that protocol 2 antibodies completely neutralize linaclotide's activity at GC-C at a molar ratio of 32/1, antibody to linaclotide (FIG. 25). The neutralization of linaclotide's activity at GC-C is dependent on concentration of antibody. Antibodies produced using protocols 1, 3, and 4 also inhibit the activity of linaclotide at GC-C in a concentration-dependent manner, but fail to completely block linaclotide's activity at the highest concentration tested (32/1 molar ratio of antibody to linaclotide). Because protocol 2 antibodies neutralize the pharmacological activity of linaclotide at GC-C in a concentration-dependent manner and can completely block this activity at the highest concentration, the rabbit polyclonal antibodies produced under protocol 2 are appropriate controls for the neutralization assay.

### Antibody Specificity

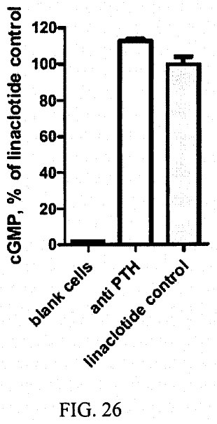

To demonstrate that the neutralization assay is specific for anti-linaclotide antibodies, anti-PTH antibodies were tested using the T84 assay (anti-PTH antibodies were used in a molar ratio of 8/1 antibody to linaclotide, linaclotide was tested at 100 nM). The anti-PTH antibodies did not inhibit linaclotide activity (FIG. 26). This lack of inhibition by an antibody directed against a non-related peptide (parathyroid hormone 1-34) demonstrated that the T84 assay is specific for antibodies that bind to linaclotide.

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed is:

1. A antibody or antigen-binding fragment thereof that binds to an epitope of linaclotide.

2. The antibody or antigen-binding fragment thereof of claim 1, wherein the epitope comprises the C-terminal tyrosine of linaclotide.

3. The antibody or antigen-binding fragment thereof of claim 1, wherein the epitope comprises the amino acid sequence: Cys Thr Gly Cys Tyr.

4. The antibody or antigen-binding fragment thereof of claim 1, wherein the antibody or antigen-binding fragment thereof is conjugated to a detectable label.

5. The antibody or antigen-binding fragment thereof of claim 4, wherein the detectable label comprises an enzyme, a radiolabel, a peptide, a linker, a fluorescent molecule, or a chemiluminescent molecule.

6. The antibody or antigen-binding fragment thereof of claim 4, wherein the detectable label is a fluorescein-containing label.

7. The antibody or antigen-binding fragment thereof of claim 4, wherein the detectable label is SULFO-TAG.

8. The antibody or antigen-binding fragment thereof of claim 4, wherein the detectable label is a peptide selected from the group consisting of Cy3, Cy5, His6, Myc-tag, GST-tag, and maltose binding protein.

9. The antibody or antigen-binding fragment thereof of claim 1, wherein the antibody is a polyclonal, monoclonal, chimeric, humanized, or human antibody.

10. The antibody or antigen-binding fragment thereof of claim 9, wherein the antibody is a polyclonal antibody.

11. The antibody or antigen-binding fragment thereof of claim 1, wherein the antigen binding fragment thereof is an Fab, F(ab′)₂, scFv, Fab′-SH, diabody, triabody, or linear antibody.

12. A method for detecting linaclotide in a biological specimen which comprises:
a) contacting the specimen with a first antibody or antigen-binding fragment thereof, wherein the first antibody is the antibody or antigen binding fragment of claim 1, thereby forming a complex between linaclotide and the first antibody or antigen-binding fragment thereof; and
b) assaying for the presence of the complex.

13. The method of claim 12, wherein the biological specimen is human plasma.

14. The method of claim 12, wherein the biological specimen is selected from the group consisting of human serum, intestinal luminal fluid, fecal matter, urine, saliva, tissue and cells.

15. The method of claim 12, wherein assaying for the presence of the complex comprises contacting the complex with a detectable label.

16. The method of claim 15, wherein the detectable label is SULFO-TAG.

17. The method of claim 15, wherein assaying for the presence of the complex comprises the addition of a second antibody to the complex, wherein said antibody specifically binds to the first antibody or antigen-binding fragment thereof in the complex.

18. The method of claim 17, wherein the second antibody is conjugated to a detectable label.

19. The method of claim 17, wherein the second antibody is an antibody conjugated with horseradish peroxidase.

20. The method of claim 18, wherein the detectable label is fluorescein.

21. A method of producing anti-linaclotide antibodies comprising:
a) conjugating linaclotide to a carrier protein;
b) immunizing an animal with the protein conjugated linaclotide to produce an immune response and thereby generate anti-linaclotide antibodies; and
c) harvesting the anti-linaclotide antibodies from the immunized animal.

22. The method of claim 21, wherein the N-terminus of linaclotide is covalently bound to the carrier protein.

23. The method of claim 21, wherein the linaclotide is reduced.

24. The method of claim 21, wherein the carrier protein is a dipalmitoyl-containing protein.

25. The method of claim 21, wherein the carrier protein is a T-Cell epitope heterodimer.

26. The method of claim 21, wherein the carrier protein is Bovine Serum Albumin (“BSA”).

27. The method of claim 21, wherein the carrier protein is Keyhole Limpet Hemocyanin (“KLH”).

28. The method of claim 21, further comprising d) purifying the anti-linaclotide antibodies.

29. The method of claim 21, wherein purifying comprises affinity chromatography.

30. A method of detecting an antibody or antigen-binding fragment of linaclotide in a biological specimen which comprises:
a) contacting the specimen with linaclotide or epitope thereof, wherein the linaclotide or epitope thereof is conjugated or bound to a detection label, tag, or substrate, thereby forming a complex between linaclotide and the antibody or antigen-binding fragment thereof of linaclotide; and
b) assaying for the presence of the complex.

31. The method of claim 30, wherein assaying for the presence of the complex comprises performing an assay selected from the group consisting of dot membrane (dot blot) assay, enzyme-linked immunosorbent assay (ELISA), Fluorescence-linked immunosorbent assay (FLISA) and electrochemiluminescence.

32. The method of claim 30, further comprising conjugating the antibody or antigen-binding fragment thereof of linaclotide with a detection label before contacting the specimen with linaclotide or epitope thereof.

33. A method for qualifying a manufacturing batch of linaclotide comprising;
a) providing a batch of linaclotide;
b) contacting at least a portion of the batch of linaclotide with anti-linaclotide antibodies or antigen-binding fragment, thereby forming a complex between linaclotide and anti-linaclotide antibodies or antigen-binding fragments thereof;
c) quantifying the presence of said complex; and
d) correlating the quantity of said complex with a known quantity of complex formed between a reference batch of linaclotide and anti-linaclotide antibodies or antigen-binding fragment thereof.

34. The method of claim 33, wherein the anti-linaclotide antibodies or antigen-binding fragment is conjugated to a detectable label.

35. The method of claim 34, wherein the detectable label comprises an enzyme, a radiolabel, a peptide, a linker, a fluorescent molecule, or a chemiluminescent molecule.
